SEQUENCE LISTING (36 sequences)

SEQ ID NO: 1 - protein, 8 aa, Artificial
  Nuclear localization signal
PKKKRKVC

SEQ ID NO: 2 - protein, 9 aa, Artificial
  Nuclear localization signal
GKKKYKLKH

SEQ ID NO: 3 - protein, 7 aa, Artificial
  Nuclear localization signal
KSKKKAQ

SEQ ID NO: 4 - protein, 10 aa, Artificial
  Nuclear Localization Signal
KELKQKQITK

SEQ ID NO: 5 - protein, 19 aa, Artificial
  Nuclear Localization Signal
NEWTLELLEELKNEAVRHF

SEQ ID NO: 6 - protein, 20 aa, Artificial
  Nuclear localization signal
RHSRIGVTRGRRARNGASRS

SEQ ID NO: 7 - protein, 9 aa, Artificial
  Nuclear localization signal
RKKRRQRRR

SEQ ID NO: 8 - protein, 12 aa, Artificial
  Nuclear localization signal
RQARRNRRRRWR

SEQ ID NO: 9 - protein, 7 aa, Artificial
  Nuclear localization signal
GKKRSKV

SEQ ID NO: 10 - protein, 7 aa, Artificial
  Nuclear localization signal
KSRKRKL

SEQ ID NO: 11 - protein, 18 aa, Artificial
  Nuclear localization system
RPAATKKAGQAKKKKLDK

SEQ ID NO: 12 - protein, 20 aa, Artificial
  Nuclear localization signal
RKKRKTEEESPLKDKAKKSK

SEQ ID NO: 13 - protein, 20 aa, Artificial
  Nuclear localization signal
KKYENVVIKRSPRKRGRPRK

SEQ ID NO: 14 - protein, 17 aa, Artificial
  Protein Transduction Domain
HIVTATYGRKKRRQRRR

SEQ ID NO: 15 - protein, 9 aa, Artificial
  Protein Transduction Domain
RKKRRQRRR

SEQ ID NO: 16 - protein, 24 aa, Artificial
  scFv-18-2 Binding Sequence
KKYIEIRKEAREAANGDSDGPSYM

SEQ ID NO: 17 - protein, 226 aa, Artificial
  scFv-18-2 Antibody
QVKLQESGAELVKPGASVKLSCKAFDYTFTTYDINWIKQRPGQGLWIGWIYPGSGNNKYNEKFKGKATLTADKSSRAAYMHLSSLTSEDSAVYFCAGGPLNMTGFDYWGQGTTVTVSSDIELTQSPSSMYASLGERVTITCKASQDINSYLSWFQQKPGKSPKTLIYRANRLVDGVPSRFSGSGSGQDYSLTISSLEYEDMGIYYCLQYDELPLTFGAGTKLEIKR

SEQ ID NO: 18 - protein, 7 aa, Artificial
  Complementarity determining regions
FTTYDIN

SEQ ID NO: 19 - protein, 17 aa, Artificial
  Complementarity determining regions
WIYPGSGNNKYNEKFKG

SEQ ID NO: 20 - protein, 10 aa, Artificial
  Complementarity determining regions
GPLNMTGFDY

SEQ ID NO: 21 - protein, 11 aa, Artificial
  Complementarity determining regions
KASQDINSYLS

SEQ ID NO: 22 - protein, 7 aa, Artificial
  Complementarity determining regions
RANRLVD

SEQ ID NO: 23 - protein, 9 aa, Artificial
  Complementarity determining regions
LQYDELPLT

SEQ ID NO: 24 - DNA, 36 bp, Artificial
  Primer
dccgggatccccaagcttcctccagtctgttgcaag

SEQ ID NO: 25 - DNA, 37 bp, Artificial
  Primer
dcaagcggccgccaatataaattgaccattcttctag

SEQ ID NO: 26 - DNA, 35 bp, Artificial
  Primer
dcggggatccttggcagaagtaggcctgaatgctc

SEQ ID NO: 27 - DNA, 35 bp, Artificial
  Primer
dgaagcggccgcctacattctctcgccaatgaacg

SEQ ID NO: 28 - DNA, 33 bp, Artificial
  Primer
cggggatccccattcagcctgcaggccacgcta

SEQ ID NO: 29 - DNA, 35 bp, Artificial
  Primer
dggggcggccgctcaattccaacaacatagggctt

SEQ ID NO: 30 - DNA, 37 bp, Artificial
  Primer
dgggggatccccgcggttcaataattatgtggactgc

SEQ ID NO: 31 - DNA, 38 bp, Artificial
  Primer
dcaagcggccgcctctttttggatgaaagacatgtttc

SEQ ID NO: 32 - DNA, 36 bp, Artificial
  Primer
dgggggatccggggtccctaaagatgaagtgttagc

SEQ ID NO: 33 - DNA, 37 bp, Artificial
  Primer
dgaagcggccgcagttatccacctcgtcccctggaag

SEQ ID NO: 34 - protein, 25 aa, Artificial
  non-binding sequence
LADSTLSEEMSQFDFSTGVQSYSYS

SEQ ID NO: 35 - DNA, 30 bp, Artificial
  Primer
dcttcgaattctgcaggtgaagctgcagga

SEQ ID NO: 36 - DNA, 28 bp, Artificial
  Primer
dgtggatcccgcggttccagcggatccg

(19) United States
(12) Patent Application Publication
(10) Pub. No.: US 2005/0220796 A1
(43) Pub. Date: Oct. 6, 2005

(54) Compositions and methods for modulating DNA repair

(76) Inventors: William S. Dynan, Martinez, GA (US); Shuyi Li, Martinez, GA (US); Yoshihiko Takeda, Martinez, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/813,977

(22) Filed: Mar. 31, 2004

(51) Int. Cl. A61K 39/395; C07K 16/30; A61K 38/17

(52) U.S. Cl. 424/155.1; 514/12; 530/388.8

(57) ABSTRACT

Compositions and methods for modulating a DNA repair process in vivo or in vitro are provided. One aspect of the disclosure provides a pharmaceutical composition including a DNA repair modulator. The DNA repair modulator includes, but is not limited to, compositions such as polypeptides, for example antibodies; modified polypeptides; and branched or unbranched aliphatic, cycloaliphatic, substituted aliphatic, aromatic hydrocarbons, or heterocyclic carbon-based compounds that associate with a DNA repair polypeptide, for example, DNA-PKcs.

### STATEMENT REGARDING FEDERALLY SPONSORED RESEARCH OR DEVELOPMENT

Aspects of the work described herein were supported, in part, by grant numbers GM35866 awarded by the National Institutes of Health and DE-FG07-99ER62875 awarded by the U.S. Department of Energy. Therefore, the U.S. government has certain rights in the invention.

### BACKGROUND

1. Technical Field

The disclosure is generally directed to methods and compositions for modulating DNA repair, more particularly, to inhibitors of DNA repair proteins and methods of their use.

2. Related Art

Radiotherapy is the most common non-surgical treatment for a variety of human cancers, and therapeutic interventions that increase the intrinsic sensitivity of tumor cells to radiation are of considerable interest. Radiotherapy, also called radiation therapy, includes the treatment of diseases, such as cancer, with ionizing radiation (IR). Ionizing radiation deposits energy that injures or destroys any cell in the area being treated by damaging its genetic material, making it impossible for the cell to continue to grow and multiply. Radiation can damage both cancer cells and normal cells; however cancer cells are more sensitive in part because they proliferate more rapidly than normal cells and in part because they often lack the cell cycle checkpoints that cause normal cells to stop proliferating until damage can be repaired.

Aside from radiotherapy, human exposure to IR also comes from environmental exposure, including exposure from cosmic, terrestrial, occupational and medical sources. Intentional exposure from radiologic dispersal devices is also a potential concern. As a result, mitigating cellular IR damage from environmental exposure can be an important therapeutic need.

The biological effects of IR exposure arise largely from its unique ability to induce DNA double-strand breaks (DSBs) (Ward, J. F. (1998) In Nickoloff, J. A. and Hoekstra, M. F. (eds) DNA Damage and Repair, Humana Press, Totowa, N.J., Vol. II, pp. 65-84). Even a single DSB per cell, if unrepaired, can lead to irreversible growth arrest or cell death (DiLeonardo, A., Linke, S. P., Clarkin, K. and Wahl, G. M. (1994) DNA damage triggers a prolonged p53-dependent G1 arrest and long-term induction of Cip1 in normal human fibroblasts, Genes Dev., 8, 2540-2551). Eukaryotic cells have evolved several DSB repair mechanisms to reduce the severity of IR damage (Pastink, A., Eeken, J. C. and Lohman, P. H. (2001) Genomic integrity and the repair of double-strand DNA breaks, Mutat. Res., 480/481, 37-50). In humans, the non-homologous end joining (NHEJ) pathway repairs most breaks within minutes of their occurrence by direct, DNA ligase-mediated end joining. An alternative repair mechanism, homologous recombination, uses an intact copy of the gene as a template for synthesis of new DNA spanning the DSB. In higher eukaryotes, homologous recombination occurs predominantly in the G2 phase of the cell cycle, when sister chromatids are available as template (Sonoda, E., Takata, M., Yamashita, Y. M., Morrison, C. and Takeda, S. (2001) Homologous DNA recombination in vertebrate cells. Proc. Natl. Acad. Sci. USA, 98, 8388-8394; Lee, S. E., Mitchell, R. A., Cheng, A. and Hendrickson, E. A. (1997) Evidence for DNA-PK-dependent and -independent DNA double-strand break repair pathways in mammalian cells as a function of the cell cycle. Mol. Cell. Biol., 17, 1425-1433).

Pharmacological inhibitors of DNA repair provide a facile approach for investigating the consequences when DNA repair proteins, for example DNA-PKcs, are present but not active. The most widely used of these compounds, wortmannin and LY294002, effectively inhibit DNA-PKcs in vivo and in vitro, but lack specificity for DNA-PKcs over related phosphatidylinositol 3-kinase family members such as ATM and ATR (23,24). Therefore, there is a need for inhibitors specific for DNA repair proteins.

Additionally, the efficacy of radiation therapy is limited by the dose that can be given without causing unacceptable harm to normal tissues. Although radiation and radiation-induced reactive oxygen species have a variety of effects on biological systems, tumor cell cytotoxicity is believed to arise primarily from induction of DNA double-strand breaks. Tumor cells are inherently susceptible to DSBs because they divide rapidly and have defects in normal systems for monitoring DNA damage. Thus, there is a need for inhibitors of DSB repair, for example inhibitors of DSB repair that increase the intrinsic sensitivity of aberrant cells to radiation-induced DSBs.

### SUMMARY

In general, the present disclosure provides compositions and methods for modulating a DNA repair process. DNA repair processes include, but are not limited to, homologous recombination, non-homologous end-joining, and single strand annealing. One aspect of the disclosure provides a pharmaceutical composition including a DNA repair modulator. The DNA repair modulator includes, but is not limited to, compositions such as polypeptides, for example antibodies; modified polypeptides; and branched or unbranched aliphatic, cycloaliphatic, substituted aliphatic, aromatic hydrocarbons, or heterocyclic carbon-based compounds that associate with a DNA repair polypeptide, for example, DNA-PKcs. Exemplary DNA repair modulators interfere with a DNA repair process, for example non-homologous end joining, resulting in the persistence of double-strand breaks (DSBs). This interference is generally accomplished by binding to a region of a DNA repair polypeptide, for example a regulatory site (outside of the catalytic domain.) in DNA-PKcs, Such binding can prevent or reduce the formation of DNA repair complexes necessary to repair DSBs, for example by interfering with protein-protein interactions.

Another aspect of the disclosure provides a DNA repair modulator targeting DNA-PKcs activity. One such modulator includes, but is not limited to, a polypeptide such as a single chain antibody variable fragment (scFv). In a particular aspect, the scFv recognizes a 25 residue linear peptide unique to DNA-PKcs, outside the conserved protein kinase catalytic domain. The scFv sensitizes cells to radiation by altering DSB repair in situ in living cells. Methods for screening for DNA repair modulators and for treating cancer are also provided.

Still other aspects of the disclosure provide DNA repair modulators including a polypeptide that specifically binds to a region of DNA-PKcs outside of the catalytic domain, wherein the polypeptide is operably linked to a targeting sequence. The targeting sequence can be an organelle localization signal. Representative organelle localization signals include, but are not limited to, nuclear localization signals, chloroplast localization signals, and mitochondrion localization signals. The targeting signals direct the polypeptide to a specific organelle or intracellular location, typically to a location of DNA repair enzymes, such as the nucleoplasm. The DNA repair modulators can also be operably linked to a protein transduction domain (PTD) to facilitate introduction of the DNA repair modulators into a cell.

Other aspects of the disclosure include methods of identifying DNA repair modulators that bind to DNA repair proteins, for example outside of the catalytic domain. Additionally, vectors encoding the disclosed DNA repair modulators and the cells transfected with these vectors are provided.

Yet another aspect provides methods for sensitizing a cell to ionizing radiation by contacting the cell with a DNA repair modulator, for example a polypeptide that specifically binds to a region of DNA-PKcs outside of the catalytic domain, in an amount sufficient to inhibit repair of double-strand breaks in the cell's DNA.

Other systems, methods, features, and advantages of the present disclosure will be or become apparent to one of skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

FIG. 1A is gel showing SDS-PAGE separation of crude periplasmic extract after affinity purification of scFv.

FIG. 1B is graph showing scFv 18-2 ligand interaction measured by surface plasmon resonance.

FIG. 1C is an immunoblot showing that scFv 18-2 binds selectively to the N-terminal fragment of DNA-PKcs.

FIGS. 1D and 1E are a series of gels showing SDS-PAGE separation of immunoprecipitates for epitope mapping of scFv 18-2.

FIG. 1F is a bar graph showing selective binding of scFv 18-2 to a peptide representing residues 2001-2025 of DNA-PKcs.

FIG. 1G is graph showing that scFv 18-2 binds to the peptide representing residues 2001-2025 of DNA-PKcs as determined by plasmon resonance.

FIG. 1H is a diagram showing fragments of DNA-PKcs used for epitope mapping of scFv 18-2.

FIG. 2A is an autoradiograph and bar graph showing scFv 18-2 inhibits the DNA end joining reaction in a cell-free assay system.

FIG. 2B is a bar graph showing scFv 18-2 only partially inhibits DNA-PKcs phosporylation of a p53 peptide substrate, which is a standardly used assay of kinase activity.

FIG. 3A is a panel of fluorescence micrographs showing cells microinjected with scFv 18-2.

FIG. 3B is a panel of phase contrast micrographs showing cells microinjected with scFv 18-2 and treated with 0 or 1.5 Gy of ionizing radiation, compared to cells microinjected with control scFv.

FIG. 3C is a panel of fluorescence micrographs showing that cells injected with scFv 18-2 and surviving irradiation are positive for active caspase 3.

FIG. 4A is a panel of fluorescence micrographs of SK-MEL-28 cells injected with scFv 18-2, irradiated with 0 or 1.5 Gy, and stained with DAPI, anti-γ-H2AX antibody and anti-GFP antibody.

FIG. 4B is a panel of fluorescence micrographs of SK-MEL-28 cells injected with scFv 18-2, treated with 0 or 0.1 Gy of ionizing radiation, and stained with DAPI, anti-γ-H2AX antibody and anti-GFP antibody.

FIG. 5A is a diagram of cDNA for scFv 18-2 inserted in-frame upstream of the EGFP gene to create a hybrid gene encoding a fusion protein, referred to hereafter as “18-2-EGFP.”.

FIG. 5B is a panel of fluorescence micrographs showing expression of 18-2-EGFP in human melanoma cells.

FIG. 6 is a panel of fluorescence micrographs of SK-MEL-28 cells transfected with the 18-2-EGFP expression construct or with the parental vector, pEGFP-N1 as indicated. The panel demonstrates the prolonged lifetime of γ-H2AX foci in 18-2-EGFP-expressing cells.

FIG. 7 is a panel of fluorescence micrographs showing that intracellular expression of 18-2-EGFP inhibits recruitment of 53BP1.

### DETAILED DESCRIPTION

Generally, the embodiments of the present disclosure are directed to compositions and methods for modulating polynucleotide repair, in particular, DNA repair. Prior to describing the various embodiments of the disclosure in detail, definitions of certain terms are provided.

1. Definitions

The term “DNA repair modulator” means an inhibitor or activator of a polynucleotide repair process. Representative DNA repair modulators sterically interfere with DNA repair processes by binding to a polypeptide or polynucleotide involved in repair processes.

The term “nuclear localization signal” or “NLS” includes, but is not limited to, polypeptides or modified polypeptides that facilitate translocation of a substance into the nucleus. Representative NLS include, but are not limited to, Large T (PKKKRKVC) (SEQ. ID NO.:1); MA-NLS1 (GKKKYKLKH) (SEQ. ID NO.:2); MA-NLS2 (KSKKKAQ) (SEQ. ID NO.:3); IN-NLS (KRK and KELKQKQITK) (SEQ. ID NO.:4); Vpr N (NEWTLELLEELKNEAVRHF) (SEQ. ID NO.:5); Vpr C (RHSRIGVTRGRRARNGASRS) (SEQ. ID NO.:6); Tat-NLS (RKKRRQRRR) (SEQ. ID NO.:7); Rev NLS (RQARRNRRRRWR) (SEQ. ID NO.:8). H2B (GKKRSKV) (SEQ. ID NO.:9); v-Jun (KSRKRKL) (SEQ. ID NO.:10) nucleoplasmin (RPAATKKAGQAKKKKLDK) (SEQ. ID NO.:11); NIN2 (RKKRKTEEESPLKDKAKKSK) (SEQ. ID NO.:12); or SWI5 (KKYENVVIKRSPRKRGRPRK) (SEQ ID NO.:13). It will be appreciated that the NLS can be selected from those listed in NLSdb available at (http://cubic.bioc.columbia.edu/db/NLSdb/) which is incorporated by reference in its entirety.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Lesk, A. M., Ed. (1988) Computational Molecular Biology, Oxford University Press, New York; Smith, D. W., Ed. (1993) Biocomputing: Infomatics and Genome Projects. Academic Press, New York; Griffin, A. M., and Griffin, H. G., Eds. (1994) Computer Analysis of Sequence Data: Part I, Humana Press, New Jersey; von Heinje, G. (1987) Sequence Analysis in Molecular Biology, Academic Press; Gribskov, M. and Devereux, J., Eds. (1991) Sequence Analysis Primer. M Stockton Press, New York; Carillo, H. and Lipman, D. (1988) SIAM J Applied Math., 48, 1073).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, ((1970) J. Mol. Biol., 48, 443-453) algorithm (e.g., NBLAST, and XBLAST). The default parameters are used to determine the identity for the polypeptides of the present invention.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating said symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

“Localization Signal or Sequence or Domain” or “Targeting Signal or Sequence or Domain” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location. Exemplary organelle localization signals include nuclear localization signals known in the art and other organelle localization signals known in the art such as those described in Emanuelson et al. (2000) Predicting Subcellular Localization of Proteins Based on Their N-terminal Amino Acid Sequence. Journal of Molecular Biology., 300, (4), 1005-1016, and in Cline and Henry (1996) Import and Routing of Nucleus-encoded Chloroplast Proteins. Annual Review of Cell & Developmental Biology, 12, 1-26, the disclosures of which are incorporated herein by reference in their entirety. It will be appreciated that the entire sequence need not be included, and modifications including truncations of these sequences are within the scope of the invention provided the sequences operate to direct a linked molecule to a specific organelle, cell, or tissue. For example, organelle localization signals include signals having or conferring a net charge, for example a positive charge. Positively charged signals can be used to target negatively charged organelles such as the mitochondria. Negatively charged signals can be used to target positively charged organelles.

“Protein Transduction Domain” or PTD refers to a polypeptide, polynucleotide, carbohydrate, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. Exemplary PTDs include but are not limited to HIV TAT YGRKKRRQRRR (SEQ. ID NO.:13) or RKKRRQRRR (SEQ. ID NO.:14); 11 Arginine residues, or positively charged polypeptides or polynucleotides having 8-15 residues, preferably 9-11 residues.

As used herein, the term “exogenous DNA” or “exogenous nucleic acid sequence” or “exogenous polynucleotide” refers to a nucleic acid sequence that was introduced into a cell or organelle from an external source. Typically the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “vector” is used in reference to a vehicle used to introduce a nucleic acid sequence into a cell. A viral vector is virus that has been modified to allow recombinant DNA sequences to be introduced into host cells or cell organelles.

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term “nucleic acid” or “nucleic acid sequence” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

“Oligonucleotide(s)” refers to relatively short polynucleotides. Often the term refers to single-stranded deoxyribonucleotides, but it can refer as well to single-or double-stranded ribonucleotides, RNA:DNA hybrids and double-stranded DNAs, among others.

“Steric inhibition” means whole or partial inhibition caused by physically blocking, masking, or making unavailable a biologically active region or component of a biologically system including a DNA repair system. Steric inhibition can be accomplished by inhibiting protein-protein interactions, for example by inducing a conformational or structural change in a DNA repair polypeptide.

The term “prodrug” refers to an agent, including nucleic acids and proteins, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. (Harper, N. J. (1962) Drug Latentiation in Jucker, ed. Progress in Drug Research, 4, 221-294; Morozowich et al. (1977) Application of Physical Organic Principles to Prodrug Design in E. B. Roche ed. Design of Biopharmaceutical Properties through Prodrugs and Analogs, APHA; Acad. Pharm. Sci.; E. B. Roche, ed. (1977) Bioreversible Carriers in Drug in Drug Design, Theory and Application, APhA, H; Bundgaard, ed. (1985) Design of Prodrugs, Elsevier; Wang et al. (1999) Prodrug approaches to the improved delivery of peptide drug, Curr. Pharm. Design, 5, 4, 265-287; Pauletti et al. (1997) Improvement in peptide bioavailability: Peptidomimetics and Prodrug Strategies, Adv. Drug. Delivery Rev, 27, 235-256; Mizen et al. (1998) The Use of Esters as Prodrugs for Oral Delivery of β-Lactam antibiotics, Pharm. Biotech, 11, 345-365; Gaignault et al. (1996) Designing Prodrugs and Bioprecursors I. Carrier Prodrugs, Pract. Med. Chem. 671-696; M. Asgharnejad (2000) Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15, 2, 143-53; Balimane and Sinko (1999) Involvement of multiple transporters in the oral absorption of nucleoside analogues, Adv. Drug Delivery Rev., 39, 1-3,183-209; Browne (1997) Fosphenyloin (Cerebyx), Clin. Neuropharmacol., 20, 1, 1-12; Bundgaard (1979) Bioreversible derivatization of drugs—principle and applicability to improve the therapeutic effects of drugs, Arch. Pharm. Chemi., 86, 1, 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev., 19, 2, 115-130; Fleisher et al. (1985) Design of prodrugs for improved gastrointestinal absorption by intestinal enzyme targeting, Methods Enzymol., 112, 360-81; Farquhar D, et al. (1983) Biologically Reversible Phosphate-Protective Groups, J. Pharm. Sci., 72, 3, 324-325; Han, H. K. et al. (2000) Targeted prodrug design to optimize drug delivery, AAPS PharmSci., 2, 1, E6; Sadzuka Y. (2000) Effective prodrug liposome and conversion to active metabolite, Curr Drug Metab., 1, 1, 31-48; D. M. Lambert (2000) Rationale and applications of lipids as prodrug carriers, Eur. J. Pharm. Sci., 11 Suppl 2, S15-27; Wang, W. et al. (1999) Prodrug approaches to the improved delivery of peptide drugs. Curr. Pharm. Des., 5, 4,265-87).

2. DNA Repair Modulators

Embodiments of the present disclosure include DNA repair modulators, for example polypeptide modulators. Exemplary polypeptide DNA repair modulators bind to a DNA repair polypeptide and inhibit DNA repair, for example non-homologous end joining. DNA repair processes in mammals, for example humans, include the non-homologous end joining (NHEJ) pathway which repairs most breaks within minutes of their occurrence by direct, DNA ligase-mediated end joining. An alternative repair mechanism, homologous recombination, uses an intact copy of the gene as a template for synthesis of new DNA spanning the DSB. In higher eukaryotes, homologous recombination occurs predominantly in the G2 phase of the cell cycle, when sister chromatids are available as template (Sonoda, E., Takata, M., Yamashita, Y. M., Morrison, C. and Takeda, S. (2001) Homologous DNA recombination in vertebrate cells. Proc. Natl. Acad. Sci. USA, 98, 8388-8394; Lee, S. E., Mitchell, R. A., Cheng, A. and Hendrickson, E. A. (1997) Evidence for DNA-PK-dependent and -independent DNA double-strand break repair pathways in mammalian cells as a function of the cell cycle. Mol. Cell. Biol., 17, 1425-1433).

Although not all components of the NHEJ system have been identified, the DNA-dependent protein kinase (DNA-PK) is known to play a central role. Accordingly, another embodiment provides DNA repair modulators that specifically bind to DNA-PK. This enzyme is composed of a regulatory component, Ku protein, and the DNA-dependent protein kinase catalytic subunit (DNA-PKcs), which bind cooperatively to free DNA ends to form an active protein kinase complex (Dvir, A., Peterson, S. R., Knuth, M. W., Lu, H. and Dynan, W. S. (1992) Ku antoantigen is the regulatory component of a template-associated protein kinase that phosphorylates RNA polymerase II. Proc. Natl. Acad. Sci. USA, 89, 11920-11924; Gottlieb, T. M. and Jackson, S. P. (1993) The DNA-dependent protein kinase: requirement for DNA ends and association with Ku antigen. Cell, 72, 131-142). DNA-PKcs phosphorylates itself, other repair proteins and p53 (Smith, G. C. and Jackson, S. P. (1999) The DNA-dependent protein kinase. Genes Dev., 13, 916-934). In rodents, DNA-PKcs mutants show greatly increased sensitivity to IR (Taccioli, G. E., Amatucci, A. G., Beamish, H. J., Gell, D., Xiang, X. H., Torres Arzayus, M. I., Priestley, A., Jackson, S. P., Marshak Rothstein, A., Jeggo, P. A. et al (1998) Targeted disruption of the catalytic subunit of the DNA-PK gene in mice confers severe combined immunodeficiency and radiosensitivity. Immunity, 9, 355-366; Gao Y., Chaudhuri, J., Zhu, C., Davidson, L., Weaver, D. T., and Alt, F. W. (1998) A targeted DNA-PKcs-null mutation revels DNA-PK-independent functions for KU in V(D)J recombination. Immunity, 9, 367-376) and in human tumors, there is an inverse correlation between the level of DNA-PKcs and radiation sensitivity (Vaganay-Juery, S., Muller, C., Marangoni, E., Abdulkarim, B., Deutsch, E., Lambin, P., Calsou, P., Eschwege, F., Salles, B., Joiner, M. et al (2000) Decreased DNA-PK activity in human cancer cells exhibiting hypersensitivity to low-dose irradiation. Br. J. Cancer, 83, 514-518.). The radiosensitive phenotype of mutant cells can be rescued by introduction of a functional DNA-PKcs cDNA, but this is not seen when using a DNA-PKcs point mutant that lacks kinase activity (Kurimasa, A., Kumano, S., Boubnov, N. V., Story, M. D., Tung, C. S., Peterson, S. R. and Chen, D. J. (1999) Requirement for the kinase activity of human DNA-dependent protein kinase catalytic subunit in DNA strand break rejoining. Mol. Cell. Biol., 19, 3877-3884). Thus, kinase activity itself is needed for DSB repair.

DSB repair takes place in vivo within cytologically defined foci characterized by the presence of a modified histone (γ-H2AX), autophosphorylated DNA-PKcs and a number of other signaling and repair proteins (Rogakou, E. P., Boon, C., Redon, C. and Bonner, W. M. (1999) Megabase chromatin domains involved in DNA double-strand breaks in vivo. J. Cell Biol., 146, 905-916; Paull, T. T., Rogakou, E. P., Yamazaki, V., Kirchgessner, C. U., Gellert, M. and Bonner, W. M. (2000) A critical role for histone H2AX in recruitment of repair factors to nuclear foci after DNA damage. Curr. Biol., 10, 886-895; Schultz, L. B., Chehab, N. H., Malikzay, A. and Halazonetis, T. D. (2000) p53 binding protein 1 (53BP1) is an early participant in the cellular response to DNA double-strand breaks. J. Cell Biol., 151, 1381-1390; Chan, D. W., Chen, B. P., Prithivirajsingh, S., Kurimasa, A., Story, M. D., Qin, J. and Chen D. J. (2002) Autophosphorylation of the DNA-dependent protein kinase catalytic subunit is required for rejoining of DNA double-strand breaks. Genes Dev., 16, 2333-2338; Mirzoeva, O. K. and Petrini, J. H. (2001) DNA damage-dependent nuclear dynamics of the Mre11 complex. Mol. Cell. Biol., 21, 281-288; Shang, Y. L., Bodero, A. J. and Chen, P. L (2003) NFBD1, a novel nuclear protein with signature motifs of FHA and BRCT and an internal 41-amino acid repeat sequence, is an early participant in DNA damage response. J. Biol. Chem., 278, 6323-6329; Xu, X. and Stern, D. F. (2003) NFBD1/KIAA0170 is a chromatin-associated protein involved in DNA damage signaling pathways. J. Biol. Chem., 278, 8795-8803; Lou, Z., Chini, C. C., Minter-Dykhouse, K. and Chen, J. (2003) Mediator of DNA damage checkpoint protein 1 regulates BRCA1 localization and phosphorylation in DNA damage checkpoint control. J. Biol. Chem., 278, 13599-13602.).

Two general approaches have been taken to investigate the role of DNA-PKcs within these foci, including its interaction with cellular DNA damage signaling pathways. In one of these, the expression of DNA-PKcs has been attenuated or eliminated through the use of antisense RNA, siRNA or targeted gene disruption (Taccioli, G. E., Amatucci, A. G., Beamish, H. J., Gell, D., Xiang, X. H., Torres Arzayus, M. I., Priestley, A., Jackson, S. P., Marshak Rothstein, A., Jeggo, P. A. et al (1998) Targeted disruption of the catalytic subunit of the DNA-PK gene in mice confers severe combined immunodeficiency and radiosensitivity. Immunity, 9, 355-366; Gao Y., Chaudhuri, J., Zhu, C., Davidson, L., Weaver, D. T., and Alt, F. W. (1998) A targeted DNA-PKcs-null mutation revels DNA-PK-independent functions for KU in V(D)J recombination. Immunity, 9, 367-376; Sak, A., Stuschke, M., Wurm, R., Schroeder, G., Sinn, B., Wolf, G. and Budach, V. (2002) Selective inactivation of DNA-dependent protein kinase with antisense oligodeoxynucleotides: consequences for the rejoining of radiation-induced DNA double-strand breaks and radiosensitivity of human cancer cell lines. Cancer Res., 62, 6621-6224; Peng, Y., Zhang, Q., Nagasawa, H., Okayasu, R., Liber, H. and Bedford, J. (2002) Silencing expression of the catalytic subunit of DNA-dependent protein kinase by small interfering RNA sensitizes human cells for radiation-induced chromosome damage, cell killing and mutation. Cancer Res., 62, 6400-6404).

In the other of these pharmacological inhibitors provide a more facile approach for investigating the consequences when DNA-PKcs is present but not active. The most widely used of these compounds, wortmannin and LY294002, effectively inhibit DNA-PKcs in vivo and in vitro, but lack specificity for DNA-PKcs over related phosphatidylinositol 3-kinase family members such as ATM and ATR (23,24). This again limits the utility of the approach.

One of the several embodiments of the present disclosure provides a pharmaceutical composition including a DNA repair modulator, a prodrug thereof, or combination thereof. Exemplary DNA repair modulators include substances that associate or bind to a DNA repair polypeptide. For example, a modulator in one embodiment of the present disclosure inhibits DNA repair by interacting with polypeptides involved in repairing double-strand breaks in DNA. One exemplary polypeptide involved in repairing DNA double strand breaks (DSB) includes, but is not limited to, DNA-PKcs. In this embodiment, the interaction of the modulator with a DNA-PKcs polypeptide. is outside of the DNA-PKcs catalytic domain. In another embodiment, the modulator binds to or associates with a region of DNA-PKcs having the sequence KKYIEIRKEAREAANGDSDGPSYM (SEQ. ID. NO.:16). Optionally, the DNA repair modulator can inhibit the kinase activity of the DNA-PKcs polypeptide. Suitable DNA repair modulators that bind outside the catalytic domain of a DNA repair polypeptide include, but are not limited to, polypeptides; antibodies; carbohydrates; peptide nucleic acids; chemically modified polypeptides having for example modified linkages; branched or unbranched aliphatic; cycloaliphatic; substituted aliphatic; aromatic hydrocarbons; or heterocyclic carbon-based compounds. Other embodiments of the present disclosure provide DNA repair modulators that do not inhibit a kinase activity of a DNA repair polypeptide, for example no more than 50%, typically not more than 20%, more typically not more than 10% inhibition.

2.1 Antibodies as DNA Repair Modulators

Another embodiment of the present disclosure provides an antibody or a fragment thereof as a DNA repair modulator. The disclosed antibodies or antibody fragments include intracellular antibodies that bind to their respective epitopes under intracellular conditions. Typically, the antibody DNA repair modulators bind to DNA repair polypeptides, for example DNA-PK, more particularly DNA-PKcs. In one embodiment, the binding of the antibody DNA repair modulator occurs outside of the catalytic domain of DNA-PKcs, for example in a region including the sequence KKYIEIRKEAREAANGDSDGPSYM (SEQ. ID. NO.:16).

Another embodiment provides a DNA repair modulator having the sequence QVKLQESGAELVKPGASVKLSCKAFDYTFTTYDINWIKQRPGQGLWIGWIYPGS GNNKYNEKFKGKATLTADKSSRAAYMHLSSLTSEDSAVYFCAGGPLNMTGFDY WGQGTTVTVSSDIELTQSPSSMYASLGERVTITCKASQDINSYLSWFQQKPGKSP KTLIYRANRLVDGVPSRFSGSGSGQDYSLTISSLEYEDMGIYYCLQYDELPLTFGA GTKLEIKR (SEQ. ID NO.:17) or a fragment thereof, wherein the DNA repair modulator specifically binds to DNA-PKcs, in particular to a region of DNA-PKcs outside the catalytic domain having the sequence KKYIEIRKEAREAANGDSDGPSYM, (SEQ. ID NO.:16) optionally including a nuclear localization signal and/or a PTD, or a combination thereof.

Yet another embodiment provides a single chain antibody that specifically binds to DNA-PKcs in a region of outside of the catalytic domain, wherein the single chain antibody includes complementarity determining regions FTTYDIN (SEQ. ID NO.:18), WIYPGSGNNKYNEKFKG (SEQ. ID NO.:19), GPLNMTGFDY (SEQ. ID NO.:20), KASQDINSYLS (SEQ. ID NO.:21), RANRLVD (SEQ. ID NO.:22), LQYDELPLT (SEQ. ID NO.:23), in an immunoglobin framework.

Without being bound to any one theory, it is believed that certain embodiments disclose DNA repair modulators that inhibit DNA repair by binding to the sequence KKYIEIRKEAREAANGDSDGPSYM (SEQ. ID NO.:16) and inducing a conformational change in DNA-PKcs that results in the steric inhibition of DSB repair. Thus, the present disclosure encompasses DNA repair modulators that specifically bind the sequence KKYIEIRKEAREAANGDSDGPSYM (SEQ. ID NO.:16). It will be appreciated by those of skill in the art that the DNA modulators of one embodiment of the present disclosure can bind to any region outside of a catalytic domain so long as the binding results in the inhibition of the DNA repair polypeptide, for example by inducing a conformational change in the DNA repair polypeptide.

Still another embodiment provides an scFv that specifically binds DNA-PKcs and blocks the NHEJ pathway of DSB repair by blocking end joining, either in an in vitro system based on a cell-free extract and/or in situ in living cells. Unlike known pharmacologic inhibitors of DNA-PKcs, some embodiments of the disclosed scFv do not target the conserved kinase domain of DNA-PKcs, but rather a unique site near the middle of the primary amino acid sequence KKYIEIRKEAREAANGDSDGPSYM (SEQ. ID NO.:16), within a region of previously undefined function. This sequence is unique to DNA-PKcs and is not present in related DNA damage-responsive kinases, ATM and ATR.

Yet another embodiment provides an scFv that produces only modest inhibition of kinase activity, although it can produce a complete inhibition of end joining. Inhibition of kinase activity can be controlled by the binding location of the antibody to the DNA repair polypeptide. Thus, blockage of DSB repair is not attributable to loss of kinase activity per se, but rather to some other mechanism. In vitro studies suggest that the antibody DNA modulator does not block DNA interaction, as there is no change in crosslinking of DNA-PKcs to photoreactive DNAs in vitro. Consistent with this, residual kinase activity seen in the presence of mAb 18-2 remains DNA-dependent (Carter, T., Vancurova, I., Lou, W. and DeLeon, S. (1990) A DNA-activated protein kinase from HeLa cell nuclei. Mol. Cell. Biol., 10, 6460-6471). Presumably, the mechanism of action involves steric hindrance of an essential protein—protein interaction surface required for progression of end joining reaction.

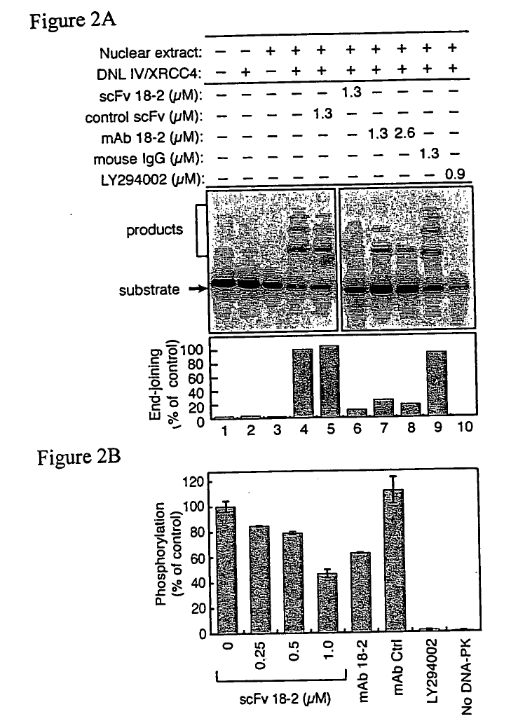

Embodiments of the disclosed scFv appear to be at least as effective at inhibiting the end joining reaction as the parental mAb, and possibly more effective, based on the comparison in FIG. 2. It could be that the smaller size of the scFv renders it better able to interact with its epitope in native DNA-PKcs. The scFv is from a recombinant source and is therefore homogeneous, whereas the mAb is purified from cell culture supernatant and could be contaminated with other IgGs from the growth medium. Generally, the mAb 18-2-producing line is not amenable to growth in serum-free medium.

Cytologic studies suggest that DSB end joining occurs within the context of repair foci characterized by a distinctive histone phosphorylation event, by the accumulation of phosphorylated DNA-PKcs and by the recruitment of repair and signaling proteins to break site, including 53BP1, NFBD1/MDC1 and the chromatin-bound form of the Mre11∘Rad50∘NBS1 complex. The order of assembly of the proteins, the number of copies that are present and their interactions within the foci remain largely unknown. The ability to block the repair process in situ at a specific stage, using an scFv directed against an epitope in an individual repair protein, provides an opportunity to dissect the process of assembly of repair foci. For example, the appearance of histone γ-H2AX foci precedes the step blocked by scFv 18-2, whereas dephosphorylation of γ-H2AX occurs subsequent to this step.

The effect of scFvs on histone γ-H2AX foci provides direct evidence for a role of DNA-PKcs in the low dose radiation response. Prior work demonstrating a requirement for DNA-PKcs has used high, cytotoxic doses of radiation. Most human exposure, however, is to low doses, and the relevance of DNA-PKcs to the low dose response has not been established. One recent study showed that human cells with a mutant form of DNL IV were impaired in the ability to resolve histone γ-H2AX foci, implicating NHEJ as a significant mechanism of repair at low doses.

scFvs vary widely in their intracellular stability, apparently because of differences in the ability to fold in the intracellular environment (Cattaneo, A. and Biocca, S. (1999) The selection of intracellular antibodies. Trends Biotechnol., 17, 115-121.). Strategies have been described for reengineering of scFvs for more efficient intracellular expression (Jermutus, L., Honegger, A., Schwesinger, F., Hanes, J. and Pluckthun, A. (2001) Tailoring in vitro evolution for protein affinity or stability. Proc. Natl. Acad. Sci. USA, 98, 75-80.). Blockade of the NHEJ pathway in the RPE cells was effectively irreversible, as the cells that received a combination of scFv 18-2 and radiation underwent apoptosis. In vitro studies suggest that the scFv blocks progression of the end joining reaction without interfering with the DNA-binding capability of DNA-PKcs. One embodiment provides compositions and methods for the induction of an arrested DNA-PKcs complex at DNA termini. The arrested DNA-PKcs complex is potentially a more effective strategy for tumor cell radiosensitization than reduction of the expression of DNA-PKcs itself because the presence of a non-functional repair complex may block access by proteins associated with other pathways of DSB repair, creating a persistent unrepaired DSB. Whereas persistent DSBs may be tolerated in quiescent normal tissues, even a small number of unrepaired DSBs in proliferating tumor cells would be expected to lead to generation of acentric chromosomal fragments, loss of essential genes and cell death.

2.2 Modified DNA Repair Modulators

The DNA repair modulators of the present disclosure can be modified to facilitate translocation and/or expression of the DNA repair modulators to or in a specific area, cell, tissue, or organ of a host. For example, DNA repair modulators can be operably linked to a target localization signal, a protein transduction domain, or both. In one embodiment, a DNA repair modulator, for example a scFv, is operably linked to a nuclear localization signal (NLS). NLS are known in the art and include, but are not limited to, Large T (PKKKRKVC) (SEQ. ID NO.:1); MA-NLS 1 (GKKKYKLKH) (SEQ. ID NO.:2); MA-NLS2 (KSKKKAQ) (SEQ. ID NO.:3); IN-NLS (KRK and KELKQKQITK) (SEQ. ID NO.:4); Vpr N (NEWTLELLEELKNEAVRHF) (SEQ. ID NO.:5); Vpr C(RHSRIGVTRGRRARNGASRS) (SEQ. ID NO.:6); Tat-NLS (RKKRRQRRR) (SEQ. ID NO.:7); Rev NLS (RQARRNRRRRWR) (SEQ. ID NO.:8), H2B (GKKRSKV) (SEQ. ID NO.:9); v-Jun (KSRKRKL) (SEQ. ID NO.:10) nucleoplasmin (RPAATKKAGQAKKKKLDK) (SEQ. ID NO.:11); NIN2 (RKKRKTEEESPLKDKAKKSK) (SEQ. ID NO.:12); or SWI5 (KKYENVVIKRSPRKRGRPRK) (SEQ. ID NO.:13). It will be appreciated that the NLS can be selected from those listed in NLSdb available at (http://cubic.bioc.columbia.edu/db/NLSdb/) which is incorporated by reference in its entirety.

Another embodiment provides DNA repair modulators operably linked to a PTD. Suitable PTDs include, but are not limited to, HIV TAT YGRKKRRQRRR (SEQ. ID NO.:14) or RKKRRQRRR (SEQ. ID NO.:15); 11 Arginine residues, or positively charged polypeptides or polynucleotides having 8-15 residues, preferably 9-11 residues. PTDs help facilitate the translocation of the DNA repair modulators from extracellular regions to intracellular regions.

In still another embodiment, a DNA repair modulator is operably linked to a PTD and a NLS. It will be appreciated by those of skill in the art that the modified DNA repair modulators may be encoded by a vector and expressed in vitro or in vivo.

The DNA repair modulators of the present disclosure can be used in combination with one or more secondary therapeutic agents, preferably the delivery of therapeutic dosages of ionizing radiation. In one embodiment, the disclosed DNA modulators are administered to increase the susceptibility of a cell to the effects of ionizing radiation, for example therapeutic ionization radiation.

Therapeutic radiation is generally applied to a defined area of a patient's body which contains abnormal proliferative tissue, in order to maximize the dose absorbed by the abnormal tissue and minimize the dose absorbed by the nearby normal tissue. The disclosed DNA repair modulators can be applied to an area containing a pathology, for example, an area of a patient's body having abnormal proliferative tissue. Once administered to the desired area, the DNA repair modulators inhibit or reduce the ability of the cells and tissues in contact with the DNA repair modulators to repair breaks in cellular DNA. Thus, in one embodiment, the disclosed DNA repair modulators increase the susceptibility of a cell or tissue to ionizing radiation by inhibiting the cell or tissue's ability to repair DSBs in the cells DNA including, but not limited to DSBs induced by the administration of ionizing radiation.

Another embodiment provides a method for treating a tumor, for example a solid tumor, in host by contacting cells of the tumor with a composition containing a disclosed DNA repair modulator in an amount sufficient to inhibit a DNA repair process in the cells of the tumor, and exposing the cells of tumor contacted with the DNA repair modulator to an amount of ionizing radiation sufficient to induce double-strand breaks in the tumor cell's DNA which induce cell death.

Other exemplary therapeutic agents include cancer therapeutics known in the art including, but not limited to, cisplatin, a halogenated pyrimidine, fluoropyrimidines, taxol, BCNU, 5-fluorouracil, bleomycin, mitomycin, hydroxyurea, fludarabine, nucleoside analogues, topoisomerase I inhibitors, hypoxic cell sensitizers and etoposide or combinations thereof.

3. Vectors

Some embodiments of the present disclosure provided DNA repair modulators that can be expressed as encoded polypeptides or proteins. The engineering of DNA segment(s) for expression in a prokaryotic or eukaryotic system may be performed by techniques generally known to those of skill in recombinant expression. It is believed that virtually any expression system may be employed in the expression of the claimed nucleic and amino sequences.

Generally speaking, it may be more convenient to employ as the recombinant polynucleotide a cDNA version of the polynucleotide. It is believed that the use of a cDNA version will provide advantages in that the size of the gene will generally be much smaller and more readily employed to transfect the targeted cell than will a genomic gene, which will typically be up to an order of magnitude larger than the cDNA gene. However, the inventor does not exclude the possibility of employing a genomic version of a particular gene where desired.

As used herein, the terms “engineered” and “recombinant” cells are intended to refer to a cell into which an exogenous DNA segment or gene, such as a cDNA or gene has been introduced. Therefore, engineered cells are distinguishable from naturally occurring cells which do not contain a recombinantly introduced exogenous DNA segment or gene. Engineered cells are thus cells having a gene or genes introduced through the hand of man. Recombinant cells include those having an introduced cDNA or genomic DNA, and also include genes positioned adjacent to a promoter not naturally associated with the particular introduced gene.

To express a recombinant encoded protein or peptide DNA repair modulator, whether modified with a NLS or PTD or a combination thereof, in accordance with the present disclosure one would prepare an expression vector that comprises one of the claimed polynucleotides under the control of one or more promoters. To bring a coding sequence “under the control of” a promoter, one positions the 5′ end of the translational initiation site of the reading frame generally between about 1 and 50 nucleotides “downstream” of (i.e., 3′ of) the chosen promoter. The “upstream” promoter stimulates transcription of the inserted DNA and promotes expression of the encoded recombinant protein. This is the meaning of “recombinant expression” in the context used here.

Many standard techniques are available to construct expression vectors containing the appropriate nucleic acids and transcriptional/translational control sequences in order to achieve protein or peptide expression in a variety of host-expression systems. Cell types available for expression include, but are not limited to, bacteria, such as E. coli and B. subtilis transformed with recombinant phage DNA, plasmid DNA or cosmid DNA expression vectors.

Certain examples of prokaryotic hosts are E. coli strain RR1, E. coli LE392, E. coli B, E. coli chi. 1776 (ATCC No. 31537) as well as E. coli W3110 (F-, lambda-, prototrophic, ATCC No. 273325); bacilli such as Bacillus subtilis; and other enterobacteriaceae such as Salmonella typhimurium, Serratia marcescens, and various Pseudomonas species.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using pBR322, a plasmid derived from an E. coli species. Plasmid pBR322 contains genes for ampicillin and tetracycline resistance and thus provides easy means for identifying transformed cells. The pBR322 plasmid, or other microbial plasmid or phage must also contain, or be modified to contain, promoters that can be used by the microbial organism for expression of its own proteins.

In addition, phage vectors containing replicon and control sequences that are compatible with the host microorganism can be used as transforming vectors in connection with these hosts. For example, the phage lambda may be utilized in making a recombinant phage vector that can be used to transform host cells, such as E. coli LE392.

Further useful vectors include pIN vectors and pGEX vectors, for use in generating glutathione S-transferase (GST) soluble fusion proteins for later purification and separation or cleavage. Other suitable fusion proteins are those with α-galactosidase, ubiquitin, or the like.

Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

For expression in Saccharomyces, the plasmid YRp7, for example, is commonly used. This plasmid contains the trp1 gene, which provides a selection marker for a mutant strain of yeast lacking the ability to grow in tryptophan, for example ATCC No. 44076 or PEP4-1. The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other suitable promoters, which have the additional advantage of transcription controlled by growth conditions, include the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization.

Another expression system, which has been shown to be particularly suitable for single chain antibodies, is the yeast, Pichia pastoris (Rubin et al, Molecular Immunology, 39:729; Shi X et al, Protein Expression and Purification 28:321-330).

In addition to micro-organisms, cultures of cells derived from multicellular organisms may also be used as hosts. In principle, any such cell culture is workable, whether from vertebrate or invertebrate culture. In addition to mammalian cells, these include insect cell systems infected with recombinant virus expression vectors (e.g., baculovirus); and plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing one or more coding sequences.

In a useful insect system, Autographica californica nuclear polyhedrosis virus (AcNPV) is used as a vector to express foreign genes. The virus grows in Spodoptera frugiperda cells. The isolated nucleic acid coding sequences are cloned into non-essential regions (for example the polyhedron gene) of the virus and placed under control of an AcNPV promoter (for example, the polyhedron promoter). Successful insertion of the coding sequences results in the inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., U.S. Pat. No. 4,215,051).

Examples of useful mammalian host cell lines are VERO and HeLa cells, Chinese hamster ovary (CHO) cell lines, W138, BHK, COS-7, 293, HepG2, NIH3T3, RIN and MDCK cell lines. In addition, a host cell may be chosen that modulates the expression of the inserted sequences, or modifies and processes the gene product in the specific fashion desired. Such modifications (e.g., glycosylation) and processing (e.g., cleavage) of protein products may be important for the function of the encoded protein.

Different host cells have characteristic and specific mechanisms for the post-translational processing and modification of proteins. Appropriate cell lines or host systems can be chosen to ensure the correct modification and processing of the foreign protein expressed. Expression vectors for use in mammalian cells ordinarily include an origin of replication (as necessary), a promoter located in front of the gene to be expressed, along with any necessary ribosome binding sites, RNA splice sites, polyadenylation site, and transcriptional terminator sequences. The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

The promoters may be derived from the genome of mammalian cells (e.g., metallothionein promoter) or from mammalian viruses (e.g., the adenovirus late promoter; the vaccinia virus 7.5K promoter). Further, it is also possible, and may be desirable, to utilize promoter or control sequences normally associated with the desired gene sequence, provided such control sequences are compatible with the host cell systems.

A number of viral based expression systems may be utilized, for example, commonly used promoters are derived from polyoma, Adenovirus 2, cytomegalovirus and Simian Virus 40 (SV40). The early and late promoters of SV40 virus are useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication.

In cases where an adenovirus is used as an expression vector, the coding sequences may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing proteins in infected hosts.

Specific initiation signals may also be required for efficient translation of the claimed isolated nucleic acid coding sequences. These signals include the ATG initiation codon and adjacent sequences. Exogenous translational control signals, including the ATG initiation codon, may additionally need to be provided. One of ordinary skill in the art would readily be capable of determining this need and providing the necessary signals. It is well known that the initiation codon must be in-frame (or in-phase) with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators.

In eukaryotic expression, one will also typically desire to incorporate into the transcriptional unit an appropriate polyadenylation site if one was not contained within the original cloned segment. Typically, the poly A addition site is placed about 30 to 2000 nucleotides “downstream” of the termination site of the protein at a position prior to transcription termination.

For long-term, high-yield production of recombinant proteins, stable expression is preferred. For example, cell lines that stably express constructs encoding proteins may be engineered. Rather than using expression vectors that contain viral origins of replication, host cells can be transformed with vectors controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci, which in turn can be cloned and expanded into cell lines.

A number of selection systems may be used, including, but not limited, to the herpes simplex virus thymidine kinase, hypoxanthine-guanine phosphoribosyltransferase and adenine phosphoribosyltransferase genes, in tk⁻, hgprt⁻ or aprt⁻ cells, respectively. Also, antimetabolite resistance can be used as the basis of selection for dhfr, which confers resistance to methotrexate; gpt, which confers resistance to mycophenolic acid; neo, which confers resistance to the aminoglycoside G-418; and hygro, which confers resistance to hygromycin.

It is contemplated that the isolated nucleic acids of the disclosure may be “overexpressed”, i.e., expressed in increased levels relative to its natural expression in human cells, or even relative to the expression of other proteins in the recombinant host cell. Such overexpression may be assessed by a variety of methods, including radio-labeling and/or protein purification. However, simple and direct methods are preferred, for example, those involving SDS/PAGE and protein staining or immunoblotting, followed by quantitative analyses, such as densitometric scanning of the resultant gel or blot. A specific increase in the level of the recombinant protein or peptide in comparison to the level in natural human cells is indicative of overexpression, as is a relative abundance of the specific protein in relation to the other proteins produced by the host cell and, e.g., visible on a gel.

3.1 Purification of Expressed Proteins

Further aspects of the present disclosure concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state, i.e., in this case, relative to its purity within a hepatocyte or p-cell extract. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, polyethylene glycol, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., Biochem. Biophys. Res. Comm., 76:425, 1977). It will therefore be appreciated that under differing electrophoresis conditions, the apparent molecular weights of purified or partially purified expression products may vary.

3.2 In Vivo Inhibition of DNA Repair

Another embodiment of the present invention provides compositions and methods for the intracellular expression of DNA repair modulators, for example scFv 18-2, in mammalian cells. Intracellularly expressed scFv 18-2 was discovered to be effective as a modifier of the radiation response of mammalian cells. Intracellular expression affords a more practical alternative to microinjection for scFv delivery. Intracellularly expressed scFv 18-2 tends to be present in cytoplasmic aggregates. In the microinjected population, by contrast, the majority of cells show a nuclear distribution of scFv coincident with endogenous DNA-PKcs, and only a minority showed cytoplasmic distribution (Li, S., et al. (2003) Modification of the ionizing radiation response in living cells by an scFv against the DNA-dependent protein kinase. Nucleic Acids Res., 31, 20, 5848-5857).

Intracellular expression of scFv 18-2 led to a striking redistribution of DNA-PKcs into cytoplasmic bodies. The precise underlying mechanism of this redistribution has not yet been characterized. It is possible that partially native scFv enters the nucleus and interacts with DNA-PKcs, and that the complex is translocated to the cytoplasm for degradation. Alternatively, the scFv may capture nascent DNA-PKcs during synthesis.

Intracellular expression of scFv 18-2 does not interfere with formation of γ-H2AX foci, a process that requires ATM-dependent phosphorylation of the minor H2AX histone isoform. It does, however, block resolution of these foci and prevent recruitment of 53BP1. Mice lacking 53BP1 are radiosensitive and exhibit growth and other defects (Ward, I. M., et al. (2003) p53 Binding protein 53BP1 is required for DNA damage responses and tumor suppression in mice. Mol Cell Biol, 23, 7, 2556-2563). Prior work suggests that 53BP1 cooperates with HDAC4 to induce G2 checkpoint arrest (Kao, G. D., et al. (2003) Histone deacetylase 4 interacts with 53BP1 to mediate the DNA damage response. J. Cell. Biol., 160, 7, 1017-1027), and that it may participate in one of two parallel pathways of DSB-dependent signaling (id). Thus, another embodiment of the present disclosure provides compositions and methods for blocking, inhibiting, or reducing 53BP1 recruitment to sites of DNA damage. By preventing accumulation of 53BP1 and thus interfering with the G2 checkpoint, the disclosed DNA repair modulators, for example scFv 18-2, may further potentiate the effect of repair inhibition itself.

4. Screening Assays

Another embodiment of the present invention provides a method of screening for DNA repair modulators. The screening assay includes introducing a test compound into a cell or a plurality of cells. The test compound can be any substance thought to modulate DNA repair, for example by interfering with DNA end-joining. The compound can be introduced into the cell by microinjection or can be translocated across the outer membrane of cell using a PTD, liposomes, phagocytosis, a membrane permeablizing agent or membrane fusion agent. Alternatively, the test compound may be taken up by the cell or cell culture passively or actively.

Suitable cells that can be used in the assay include primary culture cells or immortalized cell lines. Immortalized cell lines, for example fibroblast cell lines, can be obtained from commercial suppliers. The cells can be eukaryotic or prokaryotic.

The cells receiving the test compound can have double-strand breaks in their genetic material, for example DNA, or double-strand breaks can be induced in the cells. DSB can be induced chemically, enzymatically, or by exposing the cells to radiation, for example ionizing radiation.

Repair of the breaks in the genetic material of cells exposed to the test compound can be determined and compared to the ability to repair breaks in genetic material of control cells can be selected. Control cells include cells that have not been exposed to the test compound.

Another embodiment provides an in vitro screening assay. In this assay, a test compound is combined with a reaction mixture, for example in a reaction vessel. The reaction mixture includes a double strand break repair proteins, for example DNA ligase IV/XRCC4 complex, Ku protein, the DNA-dependent protein complex, or optionally a mixture of these and other proteins present in a whole-cell or nuclear extract, and a plurality of oligonucleotides, optionally with appropriate pH and ionic buffering agents known in the art. After the addition of the test compound, the presence of ligated oligonucleotides in the reaction mixture is detected and compared with ligated oligonucleotides, if any, detected in a control reaction mixture without the test compound.

Optionally, the screening assay may be adapted to a high-throughput format using approaches known in the art, for example by anchoring linear DNA substrate to a solid surface of a microwell plate, adding the reaction mixture, and measuring end-joining activity based on the ability to covalently capture onto the surface a second linear DNA substrate that has been tagged to facilitate detection, for example with a fluorophore.

Optionally, the effect of the test compound on DNA-dependent protein kinase activity can be determined. Such activity can be assessed by determining whether and to what extent the protein kinase phosphorylates its substrate, for example p53.

The test compound that results in fewer ligation products than in a control reaction mixture without the test compound and does not completely inhibit DNA-dependent protein kinase activity can be selected.

Still another embodiment provides a method for identifying DNA repair modulators including detecting whether a test compound binds to the polypeptide sequence KKYIEIRKEAREAANGDSDGPSYM (SEQ. ID NO.:16). For example, a polypeptide having the sequence KKYIEIRKEAREAANGDSDGPSYM (SEQ. ID NO.:16) can be combined with a test compound, and a test compound that selective binds the polypeptide can be selected. The selected test compound can optionally be analyzed for effects on DNA-PKcs enzyme activity. Test compounds that do not inhibit DNA-PKcs enzyme activity or inhibit less than about 10% DNA-PKcs activity can be further selected. In particular, test compounds that selectively bind KKYIEIRKEAREAANGDSDGPSYM (SEQ. ID NO.:16) and induce a conformational change and/or sterically inhibit DNA end joining can be selected. For example, test compounds that form arrested DNA-PK complexes can be selected.

Still another embodiment provides immobilizing a polypeptide including the sequence KKYIEIRKEAREAANGDSDGPSYM (SEQ. ID NO.:16) on a solid support, for example beads, pins, plastic, metal, glass, filter, synthetic polymer, or natural polymer. The immobilized polypeptide is then contacted with a test compound. Test compounds that specifically bind to the polypeptide can be eluted using techniques known in the art, for example, manipulating ionic strength of buffer solutions, manipulating solvent conditions, manipulating temperature, or a combination thereof.

5. Methods of Use

Another embodiment of the present disclosure provides a method for inducing cell death or apoptosis. In this method, a DNA repair modulator that inhibits DNA end-joining is introduced into a cell, for example a rapidly dividing cell such as a cancer cell. DSBs are then induced into the cell, for example by exposing the cell to radiation, including but not limited to, ionizing radiation. The persistence of the DSB in the cell ultimately result in the cells death or apoptosis.

Other methods of treatment include administering to a host an effective amount of a DNA repair modulator to inhibit the repair of DNA double-strand breaks, wherein the DNA repair modulator inhibits kinase activity, for example DNA-PKcs activity, in the range of 0 to 50%. Optionally, the host can then be exposed to low levels of ionization radiation, for example less than 1 Gy/min, typically about 0.5 Gy/min or less.

Embodiments of the present disclosure are particularly suited for the treatment of cancers, including, but not limited to surface cancers such as skin cancers, tumors, lung cancer, prostate cancer, colon cancer, testicular cancer, and breast cancer. Compositions including the disclosed DNA repair modulators can be applied or administered to a region of interest of a host, for example to a tumor, organ, or cancerous region, and the region of interest can be exposed to ionizing radiation, for example low levels of ionizing radiation. The area exposed to the radiation can be confined to region contacted with the DNA repair modulator to prevent exposure of healthy tissues to the ionizing radiation. The dose of ionizing radiation can be delivered in separate fractions, each modulated to provide about 3 cGy to about 250 cGy, calculated to introduce about 1 to 75 DSB per cell, but in the presence of the DNA repair modulator, typically less than 75 DSB per cell, and optimally as few as 1 DSB per cell.

One embodiment provides methods for sensitizing a cell to ionizing radiation by contacting the cell with a DNA repair modulator, for example a polypeptide that specifically binds to a region of DNA-PKcs outside of the catalytic domain, in an amount sufficient to inhibit repair of double-strand breaks in the cell's DNA. Contacting the cell with the disclosed DNA repair modulator increases the persistence of DSBs in the cell, for example DSBs induced by exposing the cell to therapeutic amounts of ionizing radiation. The persistence of DSBs in a cell induces cell death.

Yet another embodiment provides sensitizing a cell to radiation including introducing a DNA repair modulator including, but not limited to, a vector encoding a DNA repair modulator into a host's cell. The DNA repair modulator is optionally linked to a nuclear localization signal, a protein transduction domain, or a combination thereof. The vector can encode a DNA repair modulator that specifically binds to DNA-PKcs in a region containing the sequence KKYIEIRKEAREAANGDSDGPSYM (SEQ. ID NO.:16). Typically, the transfected host cell is an aberrant cell such as a diseased cell, a cancer cell, or cell targeted for destruction. After the cell has been transfected, the cell can be exposed to radiation in an amount sufficient to induce DSB in the cell's genetic material. It will be appreciated that the vector can include a targeting sequence to direct the vector to a specific tissue, organ, or cell type. Such targeting sequences are known in the art an include, but are not limited to, prostate specific antigen binding proteins such as antibodies to target the vector to prostate tissues, or asialoglycoprotein to target the vector to liver tissue.

Yet another embodiment provides a method for inhibiting DNA repair in a cell by contacting the cell with a DNA repair modulator, wherein the DNA repair modulator forms an aggresome with a DNA repair polypeptide, for example DNA-PKcs. Formation of the aggresome prevents the DNA repair polypeptide from associating with its substrate, and therefore inhibits DNA repair.

6. Administration

The compositions provided herein may be administered in a physiologically acceptable carrier to a host. Preferred methods of administration include systemic or direct administration to a cell. The compositions can be administered to a cell or patient, as is generally known in the art, for example in gene therapy applications. In gene therapy applications, the compositions are introduced into cells in order to transfect an express the DNA repair modulator and localize the DNA repair modulator to the nucleus or chloroplast. “Gene therapy” includes both conventional gene therapy where a lasting effect is achieved by a single treatment, and the administration of gene therapeutic agents, which involves the one time or repeated administration of a therapeutically effective DNA or RNA.

The DNA repair modulator compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The compositions of the present invention can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the DNA repair modulator combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Dosages and desired concentrations of the DNA repair modulators disclosed herein in pharmaceutical compositions may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

7. Materials and Methods

scFv Cloning and Expression

mRNA was purified from hybridoma cells expressing mAb 18-2 (Carter, T., Vancurova, I., Lou, W. and DeLeon, S. (1990) A DNA-activated protein kinase from HeLa cell nuclei. Mol. Cell. Biol., 10, 6460-6471) and used to generate and scFv-encoding cDNA as described (Yuan, Q., Clarke, J. R., Zhou, H. R., Linz, J. E., Pestka, J. J. and Hart, L. P (1997) Molecular cloning, expression and characterization of a functional single-chain Fv antibody to the mycotoxin zearalenone, Appl. Environ. Microbiol., 63, 263-269). Products were subcloned in PCANTAB 5 E (Amersham Biosciences, Piscataway, N.J.). A 1:1 culture of Escherichia coli containing the plasmid was grown in 2×YT medium with 100 μg/ml ampicillin and 2% glucose at 30° C. to an A600 of 0.8-1.0. Cells were collected, resuspended in the same medium lacking glucose and containing 1 mM isopropyl-γ-D-thioglactopyranoside and cultured overnight. A periplasmic extract was prepared by sequential extraction of the cell pellet with 20 ml of ice-cold TES buffer (0.2 M Tris-HCl, pH 8.0, 0.5 mM EDTA, 0.5 M sucrose) and 30 ml of 0.2×TES buffer. After 1 h, centrifugation was performed and scFv was purified from the supernatant using a 5 ml HiTrap anti-E tag column (Amersham Biosciences). In some experiments, scFv was prepared by an alternative procedure, where protein was precipitated from the periplasmic extract with (NH4)₂SO₄ (75% saturation) and subjected to Superdex 75 gel filtration chromatography (Amersham Biosciences) in buffer containing 50 mM Tris-HCl pH 7.9, 12.5 mM MgCl₂, 1 mM EDTA, 5% glycerol, 1 mM dithiothreitol and 0.1 M KCl.

Clones Encoding DNA-PKcs Fragments

Clones were obtained by reverse transcription-PCR amplification of Jurkat cell mRNA using the following primer sets: residues 411-780 d(CCGGGATCCCCAAGCTTCCTCCAGTCTGTTGCAAG) (SEQ. ID NO.:24) and d(CAAGCGGCCGCCAATATAAATTGACCATTCTTCTAG) (SEQ. ID NO.:25); residues 765-1276, d(CGGGGATCCTTGGCAGAAGTAGGCCTGAATGCTC) (SEQ. ID NO.:26) and d(GAAGCGGCCGCCTACATTCTCTCGCCAATGAACG); (SEQ. ID NO.:27) residues 1247-1761, (CGGGGATCCCCATTCAGCCTGCAGGCCACGCTA) (SEQ. ID NO.:28) and d(GGGGCGGCCGCTCAATTCCAACAACATAGGGCTT) (SEQ. ID NO.:29); residues 1734-2228, d(GGGGGATCCCCGCGGTTCAATAATTATGTGGACTGC) (SEQ. ID NO.:30) and d(CAAGCGGCCGCCTCTTTTTGGATGAAAGACATGTTTC) (SEQ. ID NO.:31); residues 2204-2714, d(GGGGGATCCGGGGTCCCTAAAGATGAAGTGTTAGC) (SEQ. ID NO.:32) and d(GAAGCGGCCGCAGTTATCCACCTCGTCCCCTGGAAG) (SEQ. ID NO.:33). The cDNAs were subclone in PCITEa(+) (Novagen, Madison, Wis.) and expressed using the TNT Coupled Rabbit Reticulocyte In Vitro [³⁵S]methionine. Immunoprecipitation was as described (Takeda, Y., Caudell, P., Grady, G., Wang, G., Suwa, A., Sharp, G. C., Dynan, W. S. and Hardin, J. A. (1999) Human RNA helicase A is a lupus autoantigen that the cleaved during apoptosis. J. Immunol., 163, 6269-6274.).

Binding Parameters for scFv-DNA-PKcs Interaction

Surface plasmon resonance measurement were made using a Biacore X instrument (Biacore, Piscataway, N.J.). Interaction between scFv 18-2 and purified DNA-PKcs was measured by amine coupling of scFv to one channel of a Biosensor chip CM-5. The other channel was used as a reference. Analyte, consisting of purified DNA-PKcs diluted in HBS-EP (10 Mm HEPES, pH 7.4, 150 mM NaCl, 3 mM EDTA, 0.005% Surfactant P20) was flowed over the chip at 30 μl/min. Interaction between scFv and peptides was measured by immobilizing the specific peptide, biotin-KKKYIEIRKEAREAANGDSDGPSYM (SEQ. ID NO.:16), in one channel of a Biosensor chip SA and a non-specific peptide, representing a nearby non-binding sequence, LADSTLSEEMSQFDFSTGVQSYSYS (SEQ. ID NO.:34), in the other channel. Analyte, consisting of scFv 18-2 diluted in HBS-EP, was flowed over the chip as above. For both experiments, regeneration between runs was with HBS-EP supplemented with 4 mM MgCl₂, 100 mM glycine, pH 2.3, and 1 M NaCl. Duplicate measurements were made at 25° C. Data were additionally double referenced and evaluated using the 1:1 interaction with the mass transfer limitation model of the BioEvaluation 3.1 software.

Functional Assays

Cell-free DNA end joining assays were performed as described (Huang, J. and Dynan, W. S. (2002) Reconstitution of the mammalian DNA double-strand break end-joining reaction reveals a requirement for an Mre11/Rad50/NBS 1-containing fraction. Nucleic Acids Res., 30, 667-674). Peptide phosphorylation assays (25 μl) contained 12.5 μM ATP, 0.5 μCi [γ-³²P]ATP, 0.4 mM biotinylated p53 peptide, 2 μg bovine serum albumin and 3 ng DNA fragment (400 bp). Reactions were preincubated at 30° C. for 5 min and then DNA-PK (1 μl) and scFv or mAb were added. Reactions were incubated at 30° C. for 15 min, 12.51 μl of termination buffer (7.5 M guanidine hydrochloride) was added and 10 μl from each reaction was spotted on a biotin capture membrane (SignaTECT DNA-PK Assay System; Promega). After washing, binding of phosphopeptide was determined by liquid scintillation counting.

Microinjection

Retinal pigment epithelial cells were immortalized with telomerase. An expression construct for the oncoprotein, adenovirus E1A, was introduced using a retroviral vector (Blint, E., Phillips, A. C., Kozlov, S., Stewart, C. L. and Vousden, K. H (2002) Induction of p57(KIP2) expression by p73beta. Proc. Natl. Acad. Sci. USA, 99, 3529-3534). Cells were grown in a 50:50 (v/v) mixture of MEM and F12 media, supplemented with 10% fetal bovine serum and antibiotics. The SK-MEL-28 human skin melanoma cell line (Carey, T. E., Takahashi, T., Resnick, L. A., Oettgen, H. and Old, L. J. (1976) Cell surface antigens of human malignant melanoma: mixed hemadsorption assays for humoral immunity to cultured autologous melanoma cells. Proc. Natl. Acad. Sci. USA, 73, 3278-3282.) was grown in DMEM supplemented with 10% fetal bovine serum and antibiotics. Cells were seeded on 175 μm CELLocate overslips (Eppendorf AG, Hamburg, Germany) and microinjected using sterile microcapillaries (Femtotips II; Eppendorf AG) mounted on an automated microinjection system (FemtoJet and InjectMan; Eppendorf AG) attached to a Zeiss Axiovert microscope. The injection mixture consisted of 1 mg/ml scFv, 15 μg/ml pEGFP-N1 DNA (Clontech, Palo Alto, Calif.), 10 mM KH₂PO₄, pH 7.4, and 75 mM KCl. Each injection was performed at a pressure of 50 hPa for 0.2 s. Unless otherwise indicated in the figure legend, cells were allowed to recover for 2-3 h at 37° C. and irradiated using a ¹³⁷Cs source (GammaCell 40 Exactor; MCS Nordion, ON) at a rate of 1 Gy/min. Successfully injected cells were identified by GFP fluorescence after 12-24 h.

Immunofluorescence Staining

Cells were fixed in 2% formaldehyde for 10 min. They were permeabilized and blocked by incubation for 1 h in phosphate-buffered saline containing 0.5% Triton X-100, 15% goat serum, 0.2% fish skin gelatin and 0.03% NaN₃. Samples were incubated with one or more of the following antibodies: 1:250 dilution of anti-E-tag (detect scFv; Amersham Pharmacia Biotech, Piscataway N.J.), 1:1000 anti-DNA-PKcs (human serum FT) (Jafri, F., Hardin, J. A. and Dynan, W. S. (2001) A method to detect particle specific antibodies against Ku and the DNA-dependent protein kinase catalytic subunit in autoimmune sera. J. Immunol. Methods, 251, 53-61.), 1:500 anti-green fluorescent protein (Novus Biologicals, Littleton, Colo. or Molecular Probes, Eugene, Oreg.), 1:200 anti-activated caspase 3 (Promega), 1:500 anti-γ-H2AX (mAb JBW301; Upstate Cell Signaling Solutions, Waltham, Mass.), and anti-53BP1 (1:500, Oncogene Research Products, Boston, Mass.). Staining was visualized using secondary antibodies of appropriate specificity conjugated to Alexa Fluor 488 or Alexa Fluor 594 (Molecular Probes).

Expression of 18-2-EGFP

The scFv 18-2 cDNA (Li, S., et al. (2003) Modification of the ionizing radiation response in living cells by an scFv against the DNA-dependent protein kinase. Nucleic Acids Res, 31, 20, 5848-5857) was PCR-amplified with two primers, d(CTTCGAATTCTGCAGGTGAAGCTGCAGGA) (SEQ. ID NO.:35) and d(GTGGATCCCGCGGTTCCAGCGGATCCG) (SEQ. ID NO.:36), and the product was inserted via topoisomerase-mediated ligation into the pCR2.1-TOPO vector (Invitrogen, Carlsbad, Calif.). Recombinant clones were isolated, the scFv coding region was excised with EcoRI and BamHII, and the resulting fragment was inserted into the multiple coding site of the pEGFP-N1 vector (BD Biosciences Clontech, Palo Alto Calif.). This construct, which expresses an scFv-18-2-EGFP fusion protein (18-2-EGFP) under control of the cytomegalovirus immediate early promoter, was transfected into SK-MEL-28 human melanoma cells using Lipofectamine 2000 (Invitrogen).

### EXAMPLES

### Example 1

### scFv 18-2 Recognizes a Site in DNA-PKcs Outside of the Catalytic Domain

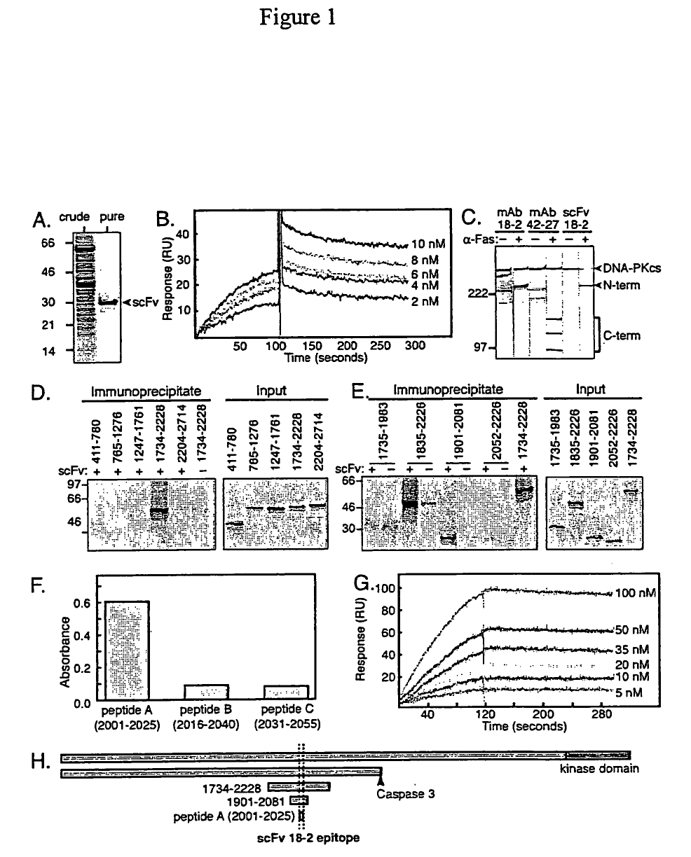

A reverse transcription-PCR strategy was used to amplify the rearranged heavy and light chain variable region genes from mAb 18-2-expressing cells. Amplified genes were assembled into a scFv-encoding cDNA, which was subcloned for overexpression in the E. coli periplasm. Purified scFv preparations, obtained by affinity chromatography as described in Material and Methods, contained a prominent 30 kDa band (FIG. 1A). This was identified as the scFv based on its size and anti-epitope tag immunoblotting. The presence of authentic heavy and light chain variable fragment sequences was verified by comparison with Kabat immunoglobulin sequence database using the AbCheck tool (Martin, A. C. (1996) Accessing the Kabat antibody sequence database by computer. Proteins, 25, 130-133) and by molecular modeling using the WAM tool (Whitelegg, N. R. and Rees, A. R. (2000) WAM: an improved algorithm for modelling antibodies on the WEB. Protein Eng., 13, 819-824).

scFv binding parameters were evaluated by surface plasmon resonance (FIG. 1B). The K_{d} was ˜1.4 nM, which is typical for antibody-antigen interactions (Siegel, R. W., Allen, B., Pavlik, P., Marks, J. D. and Bradbury, A. (2000) Mass spectral analysis of a protein complex using single-chain antibodies selected on a peptide target: applications to functional genomics. J. Mol. Biol., 302, 285-293). Immunoblotting showed the ability of scFv 18-2 to selectively recognize DNA-PKcs in a mixture of total cellular proteins (FIG. 1C) Immunoblotting also showed that the scFv epitope lies within a caspase cleavage fragment spanning residues 1-2713 (Casciola-Rosen, L., Nicholson, D. W., Chong, T., Rowan, K. R., Thornberry, N. A., Miller, D. K. and Rosen, A. (1996) Apopain/CPP33 cleaves proteins that are essential for cellular repair: a fundamental principle of apoptotic death. J. Exp. Med., 183, 1957-1964), which was produced by treating Jurkat cells with anti-Fas antibody (McConnell, K. R., Dynan, W. S. and Hardin, J. (1997) The DNA-dependent protein kinase catalytic subunit (p460) is cleaved during Fas-mediated apoptosis in Jurkat cells. J. Immunol., 158, 2083-2089). This is consistent with results of prior studies using the parental mAb (Casciola-Rosen, L., Nicholson, D. W., Chong, T., Rowan, K. R., Thornberry, N. A., Miller, D. K. and Rosen, A. (1996) Apopain/CPP33 cleaves proteins that are essential for cellular repair: a fundamental principle of apoptotic death. J. Exp. Med., 183, 1957-1964.; McConnell, K. R., Dynan, W. S. and Hardin, J. (1997) The DNA-dependent protein kinase catalytic subunit (p460) is cleaved during Fas-mediated apoptosis in Jurkat cells. J. Immunol., 158, 2083-2089). The pattern of binding differs from that of mAb 42-27, which recognizes a different epitope C-terminal to the caspase site (Song, Q., Lees-Miller, D. K., Kumar, S., Zhang, Z., Chan, D. W., et al. (1996) DNA-dependent protein kinase catalytic subunit: a target for an ICE-like protease in apoptosis, EMBO J, 15, 3238-3246).

The epitope was further delineated using overlapping cDNAs providing full coverage of the 1-2713 fragment. Proteins were expressed using a coupled in vitro transcription-translation system and scFv 18-2 binding was tested by immunoprecipitation with anti-epitope tag antibody. The epitope mapped to fragment spanning residues 1734-2228 (FIG. 1D). This sequence was further subcloned (not shown) and studies with synthetic peptides (FIG. 1F) identified at 25 residue sequence, 2001-2025, as necessary and sufficient for epitope formation. Surface plasmon resonance showed that binding parameters for interaction between scFv and the peptide were comparable to those for interaction with whole DNA-PKcs (FIG. 1G). The epitope mapping is summarized in FIG. 1H. The epitope is located outside the kinase catalytic domain, within sequences unique to DNA-PKcs and not shared with ATM or ATR.

### Example 2

### Inhibition of DNA-PK Activity in Cell-Free Assays

To test the effect of scFv 18-2 on DNA end joining, reactions were performed in a cell-free system containing linerarized plasmid substrate, HeLa cell nuclear extract and recombinant DNA ligase IV (DNA IV)/XRCC4 complex (Huang, J. and Dynan, W. S. (2002) Reconstitution of the mammalian DNA double-strand break end-joining reaction reveals a requirement for an Mre11/Rad50/NBS1-containing fraction. Nucleic Acids Res., 30, 667-674.). Consistent with previous results, nuclear extract and purified DNA IV/XRCC4 each had little activity when tested alone, but catalyzed efficient conversion of linear substrate to dimers and higher oligomeric products when tested as a mixture (FIG. 2A, lanes 1-4). scFv 18-2 strongly inhibited end joining, whereas an unrelated control scFv had little effect at an equal concentration (lanes 5 and 6). The parental mAb 18-2 inhibited end joining, although the inhibition was incomplete, even at the highest concentration tested (lanes 7 and 8). Control mouse IgG did not inhibit (lane 9). LY 294002, a relatively non-specific phosphatidylinositol 3-kinase inhibitor, blocked end joining completely under the conditions used (lane 10).

Separate assays were performed to test the effect of scFv 18-2 on kinase activity. Both the scFv and the parental mAb inhibited p53 peptide phosphorylation activity by ˜50% at the highest concentration tested (FIG. 2B), similar to results obtained in the initial characterization of mAb 18-2 (Carter, T., Vancurova, I., Lou, W. and DeLeon, S. (1990) A DNA-activated protein kinase from HeLa cell nuclei. Mol. Cell. Biol., 10, 6460-6471). The complete inhibition of end joining activity under conditions that give only partial inhibition of kinase activity is consistent with the epitope mapping results showing that the scFv 18-2 recognition sequence is outside the kinase domain.

### Example 3

### Intracellular Binding of scfv 18-2 to DNA-PKcs

Microinjection of antibodies is a well-established method to study intracellular protein function (Morgan, D. O. and Roth, R. A. (1998) Analysis of intracellular protein function by antibody injection. Immunol. Today, 9, 84-88; McNeil, P. L. (1989) Incorporation of macromolecules into living cells. Methods Cell Biol., 29, 153-173). Although scFv can, in principle, be expressed intracellularly by gene transfer, microjection was chosen for the present study because it allows introduction of native, folded antibody directly into the nucleus. This eliminates concerns over disulfide bond formation and folding in the intracellular environment, which are common obstacles to use of scFv for intracellular applications (Cattaneo, A. and Biocca, S. (1999) The selection of intracellular antibodies. Trends Biotechnol., 17, 115-121). Initial experiments were performed using telomerase-immortalized human retinal pigment epithelial (RPE) cells expressing the adenovirus E1A oncoprotein (Blint, E., Phillips, A. C., Kozlov, S., Stewart, C. L. and Vousden, K. H (2002) Induction of p57(KIP2) expression by p73beta. Proc. Natl. Acad. Sci. USA, 99, 3529-3534). These cells are sensitive to p53-mediated apoptotic signaling and are expected to respond strongly to any increase in unrepaired DSBs.

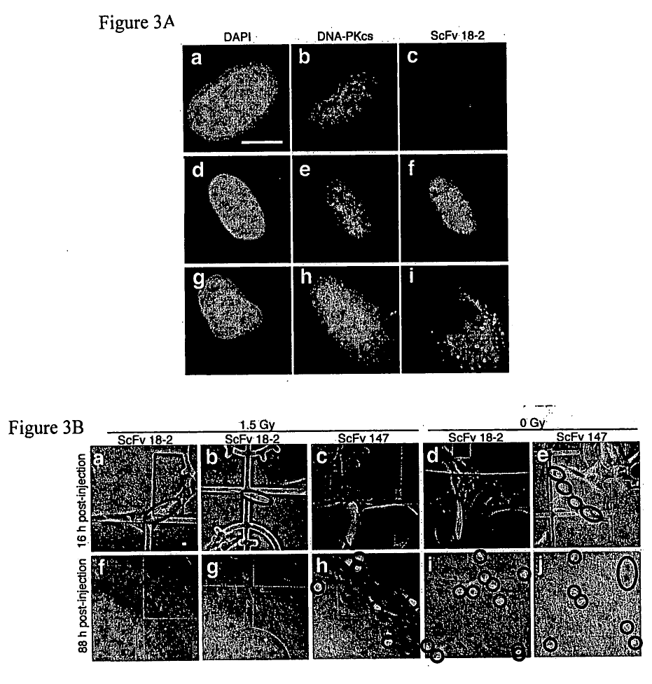

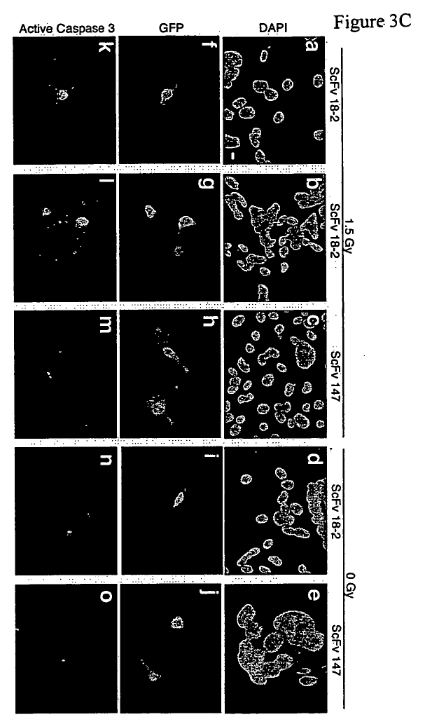

A preliminary experiment was performed to determine whether microinjected scFv 18-2 was stable and associated with DNA-PKcs in vivo. scFv was injected into the nucleus and cells were immunostained after 6 h to allow simultaneous visualization of endogenous DNA-PKcs and microinjected scFv 18-2. (FIG. 3A). In a non-injected control cell (a-e), DNA-PKcs has a punctate nuclear distribution, consistent with previous results (Mo, X., and Dynan, W. S. (2002) Subnuclear localization of Ku protein: functional association with RNA polymerase II elongation sites. Mol. Cell. Biol., 22, 8088-8099), and scFv staining is not seen. In injected cells, two patterns were seen. In the more common (f-j), DNA-PKcs retains its punctate nuclear appearance and microinjected scFv 18-2 adopts a coincident nuclear distribution. In an estimated 20% of the cells, scFv 18-2 was primarily cytoplasmic (k-o). This may reflect variability in the microinjection technique. In these cells, a portion of the DNA-PKcs appears to be drawn into the cytoplasm, where it assumes a focal distribution coincident with the scFv. Results demonstrate that scFv 18-2 is stable inside cells for at least 6 h post-injection and are consistent with binding to endogenous DNA-PKcs. In separate experiments, microinjected scFv 18-2 was detected 18 h post-injection, albeit at lower levels (not shown).

### Example 4

### Combination of scFv and IR Inhibits Microcolony Formation

The effect of microinjected scFv 18-2 colony forming ability was also investigated. Cells were co-injected with scFv and a plasmid encoding enhanced green fluorescent (EGFP), which serves as a tracer, allowing the fate of injected cells to be tracked in real time. Cells received either scFv or an unrelated control antibody, scFv 147 (Hayhurst, A. and Harris, W. J. (1999) Eschericia coli skp chaperone coexpression improves solubility and phage display of single-chain antibody fragments. Protein Expr. Purif, 15, 336-343) and received 0 or 1.5 Gy IR 6 h post-injection. The dose was chosen on the basis of preliminary experiments indicating that 1.5 Gy was somewhat below the threshold required to reduce growth or induce apoptosis in non-injected cells.

The cell growth substrate was marked with a grid pattern, permitting the same field to be observed repeatedly. At 16 h post-injection, individual microinjected cells could be recognized by intrinsic EGFP fluorescence against a background of non-injected cells, which was visualized by phase contrast illumination (FIG. 3B, a-e). There was no difference between treatment groups at this early time. However, when cells were re-observed 88 h post-injection, an estimated 60-80% of the cells that received a combination of scFv 18-2 and 1.5 Gy had disappeared from the plate (f-g) and the few remaining cells had failed to divide. Surrounding non-injected cells, visualized by DIC optics, proliferated normally. In contrast to cells in the treatment group, almost all of the cells in three control groups, which had received a combination of control scFv and 1.5 Gy, scFv 18-2 and 0 Gy, scFv 147 and 0 Gy, respectively, divided to form microcolonies of 4-8 cells (h-j).

### Example 5

### scFv Sensitizes Cells to IR-Induced Apoptosis

Non-dividing cells that remained on the coverslip after combination treatment with the scFv and 1.5 Gy IR were immunostained with antibodies against active caspase 3 (FIG. 3C). EGFP was used as a tracer to allow visualization of microinjected cells. At 60 h post-injection, the majority of cells remaining after treatment with scFv 18-2 and 1.5 Gy IR stained brightly for activated caspase 3 (FIG. 3C, k-1), whereas surrounding non-injected cells were negative (FIG. 3C, m-o). Table 1 provides a quantitation of the results. Differences in the frequency of apoptotic cells in different groups were statistically significant (P<0.001).

### Example 6

### scFv 18-2 Prevents Repair of DNA Damage

To determine whether scFv 18-2 directly inhibited repair in vivo, the fate of individual DSBs was monitored using histone γ-H2Ax as a marker. Phosphorylation of the H2A variant, H2AX, which creates the γ-H2AX form, occurs in situ within a megabase domain of chromatin flanking each DSB (Rogakou, E. P., Boon, C., Redon, C. and Bonner, W. M. (1999) Megabase chromatin domains involved in DNA double-strand breaks in vivo. J. Cell Biol., 146, 905-916). Recent studies have validated the use of γ-H2AX foci as a surrogate marker for unrepaired DSBs over a wide range of doses (Rogakou, E. P., Boon, C., Redon, C. and Bonner, W. M. (1999) Megabase chromatin domains involved in DNA double-strand breaks in vivo. J. Cell Biol., 146, 905-916; Sedelnikova, O. A., Rogakou, E. P., Panyutin, I. G. and Bonner, W. M. (2002) Quantitative detection of 125IdU-induced DNA double-strand breaks with γ-H2Ax antibody. Radiat. Res., 158, 486-492). The appearance and disappearance of γ-H2AX foci closely tracks the kinetics of DSB repair and disappearance of foci is impaired in cells that are deficient in DNL IV, an essential enzyme in the NHEJ pathway (Rogakou, E. P., Boon, C., Redon, C. and Bonner, W. M. (1999) Megabase chromatin domains involved in DNA double-strand breaks in vivo. J. Cell Biol., 146, 905-916). In preliminary experiments (not shown), prominent foci were induced in non-injected SK-MEL-28 cells in response to IR. Their appearance was does dependent, they formed within 30 min and most were resolved within 90 min.

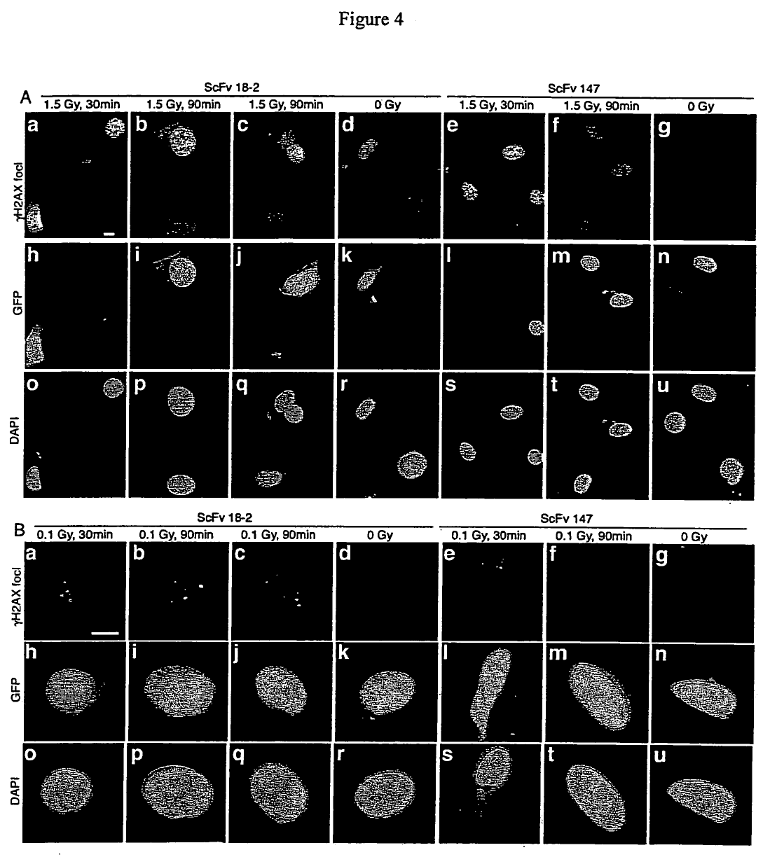

The effect of scFv 18-2 on γ-H2AX foci is shown in FIG. 4. Irradiation was performed at two doses, 1.5 and 0.1 Gy, which are calculated to induce ˜50 and ˜3 DSBs per cell, respectively, assuming a diploid genome content (Ward, J. F. (1988) DNA damage produced by ionizing radiation in mammalian cells: identities, mechanisms of formation and repairability. Prog. Nucleic Acid Res. Mol. Biol., 35, 95-125; Metzger, L. and Iliakis, G. (1991) Kinetics of DNA double-strands break repair throughout the cell cycle as assayed by pulsed field gel electrophoresis in CHO cells. Int. J. Radiat. Biol., 59, 1325-1339).

As in previous experiments, EGFP vector was co-injected to allow tracking of injected cells. At 30 min following exposure to 1.5 Gy, bright nuclear staining for γ-H2AX was seen independently of whether cells were microinjected with scFv 18-2, with control scFv or were non-injected bystanders (FIG. 4A, a and e). No staining was seen in non-irradiated control cells (d and g). At 90 min post-irradiation, the γ-H2AX persisted at high levels in cells receiving scFv 18-2 (b and c), but disappeared from non-injected cells in the same field and from cells receiving control scFv (f). FIG. 4B shows the same experiment at 0.1 Gy. Again, induction of γ-H2AX foci was similar in both irradiated groups (a and e). The foci persisted in cells receiving scFv 18-2 (b and c), but quickly resolved in non-injected cells (not shown) and in cells receiving control scFv (f). Together, these results suggest that scFv 18-2 blocks or delays repair of DSBs in vivo.

### Example 7

### Expression of scfv 18-2

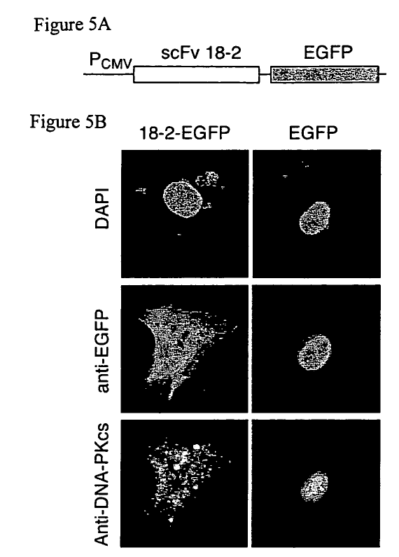

An scFv 18-2 expression construct was created by inserting the cDNA for this scFv upstream of, and in-frame with, the coding sequence for enhanced green fluorescent protein (EGFP), as described in Materials and Methods. Expression of the resulting 18-2-EGFP fusion gene was driven by the constitutive cytomegalovirus immediate early promoter. The vector was transfected into the established human melanoma cell line, SK− MEL-28, and at 24 h post-transfection, cells were fixed and stained with anti-EGFP and anti-DNA-PKcs. Results are shown in FIG. 5.

The 18-2-EGFP fusion protein accumulated to readily detectable levels. Staining was evident in both the nucleus and the cytoplasm, and in some cells the 18-2-EGFP was aggregated into discrete cytoplasmic bodies. The pattern of expression differed from that in control cells transfected with vector encoding EGFP alone, which accumulated predominantly in the nucleus. It was of interest that although 18-2-EGFP and EGFP were detected at similar levels by immunostaining of fixed cells, 18-2-EGFP showed much weaker autofluorescence than EGFP in unfixed cells (data not shown). This suggests that a significant portion of the 18-2-EGFP was unfolded, consistent with its presence in “aggresomes.” Overall, the pattern of 18-2-EGFP expression is strongly reminiscent of previous reports describing the behavior of intracellularly expressed anti-Ras scFvs (Cardinale, A., I. Filesi, and S. Biocca (2001) Aggresome formation by anti-Ras intracellular scFv fragments. The fate of the antigen-antibody complex. Eur J. Biochem., 268, 2, 268-277; Cardinale, A., et al. (1998), The mode of action of Y13-259 scFv fragment intracellularly expressed in mammalian cells., FEBS Lett., 439, 3, p. 197-202). Formation of aggresomes is an alternative mechanism by which an scFv may inhibit biological function of its target molecule.

Expression of 18-2-EGFP led to a striking redistribution of the target antigen, DNA-PKcs. In 18-2-EGFP-positive cells, DNA-PKcs was present primarily in discrete cytoplasmic bodies that partially, but not completely, overlapped the distribution of 18-2-EGFP itself. In control cells transfected with EGFP alone, there was no change in the localization of DNA-PKcs.

### Example 8

### Effect of 18-2-EGFP Expression on DSB Repair

The effect of the scFv fusion protein on DNA double-strand break repair was determined by irradiating the transfected cell population, fixing the cells after different recovery intervals, and immunostaining with anti-γ-H2AX antibody. Previous studies have shown that γ-H2AX, which is created by ATM-dependent phosphorylation of the H2AX isoform of histone H2A (Rogakou, E. P., et al. (1999) Megabase chromatin domains involved in DNA double-strand breaks in vivo. J. Cell. Biol., 146, 5, 905-916; Burma, S., et al. (2001) ATM phosphorylates histone H2AX in response to DNA double-strand breaks. J Biol. Chem., 276, 45, 42462-42467; Rogakou, E. P., et al. (1998) DNA double-strand breaks induce histone H2AX phosphorylation on serine 139. J Biol. Chem., 273, 10, 5858-5868) accumulates in nuclear foci that correspond on a 1:1 basis to unrepaired DSBs (Sedelnikova, O. A., et al. (2002) Quantitative Detection of 125IdU-Induced DNA Double-Strand Breaks with γ-H2AX Antibody. Radiat Res., 158, 486-492; Rothkamm, K. and M. Löbrich (2003) Evidence for a lack of DNA double-strand break repair in human cells exposed to very low x-ray doses. Proc. Natl. Acad. Sci. USA, 100, 5057-5062).

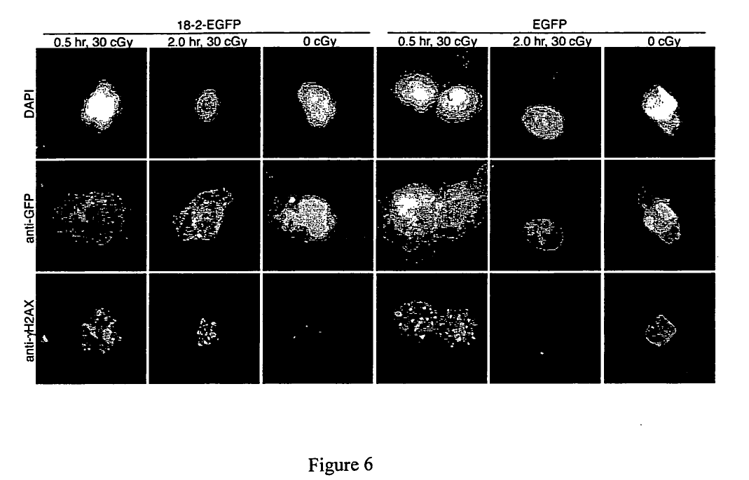

In cells transfected with either 18-2-EGFP or EGFP and treated with 30 cGy of gamma radiation, numerous γ-H2AX foci were visible after 0.5 h recovery (FIG. 6). These foci were not visible in mock-irradiated control cells. Approximately 10-20 foci were present per nucleus, consistent with the approximately 10 DSBs per diploid genome expected at this dose (Cedervall, B., et al. (1995) Methods for the quantification of DNA double-strand breaks determined from the distribution of DNA fragment sizes measured by pulsed-field gel electrophoresis. Radiat. Res., 143, 8-16; Ruiz de Almodovar, J. M., et al. (1994) A comparison of methods for calculating DNA double-strand break induction frequency in mammalian cells by pulsed-field gel electrophoresis. Int J Radiat Biol., 65, 641-649). In cells transfected with 18-2-EGFP, foci persisted at an apparently unchanged level for at least 2 h post-irradiation. By contrast, in control cells transfected with EGFP vector alone, virtually all foci had disappeared at 2 h. These results suggest that intracellularly expressed scFv 18-2 either blocks DSB repair or interferes with subsequent dephosphorylation of γ-H2AX.

### Example 9

### Failure to Recruit 53BP1

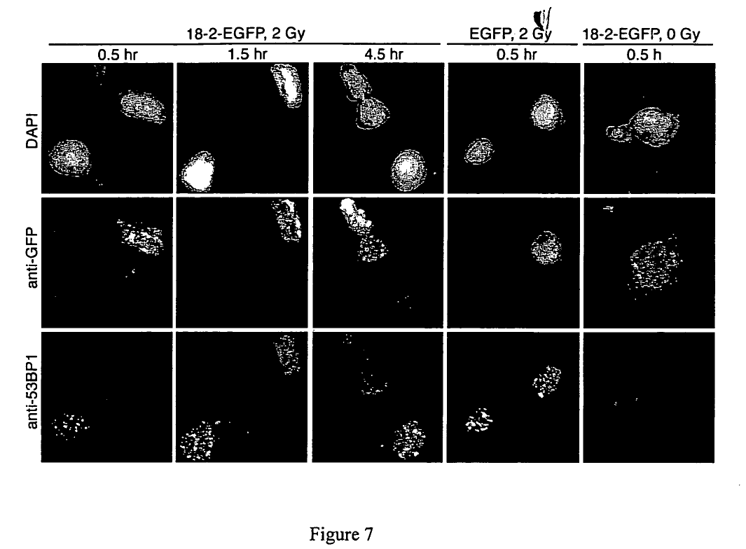

Appearance of γ-H2AX is believed to be an early step in assembly of repair foci containing a number of different proteins (Paull, T. T., et al. (2000) A critical role for histone H2AX in recruitment of repair factors to nuclear foci after DNA damage. Curr. Biol., 10, 886-895). Among these other proteins is 53BP1, which binds the tumor suppressor p53 and is involved in an HDAC4-dependent DNA damage signaling pathway (Schultz, L. B., et al., (2000) p53 binding protein 1 (53BP1) is an early participant in the cellular response to DNA double-strand breaks. J. Cell. Biol., 151, 1381-1390; Anderson, L., C. Henderson, and Y. Adachi (2001) Phosphorylation and rapid relocalization of 53BP1 to nuclear foci upon DNA damage. Mol. Cell. Biol., 21, 1719-1729; Kao, G. D., et al. (2003) Histone deacetylase 4 interacts with 53BP1 to mediate the DNA damage response. J. Cell. Biol., 160, 1017-1027). To more precisely understand the functional defect in DSB repair foci in 18-2-EGFP-transfected cells, an experiment was performed in which cells were irradiated, fixed, and immunostained for 53BP1. Results are shown in FIG. 7.

Expression of 18-2-EGFP led to a substantial inhibition of 53BP1 recruitment. Little or no accumulation of 53 BP1 in repair foci was seen at times up to 4.5 h post-irradiation. By contrast, non-transfected cells in the same field showed marked accumulation of 53BP1 within 30 min following irradiation. Cells transfected with EGFP alone also showed accumulation of 53BP1 within 30 min. The results confirm that 18-2-EGFP-expressing cells are characterized by the appearance of defective DSB repair foci in response to ionizing radiation treatment.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the invention or inventions. All such modifications and variations are intended to be included herein within the scope of this disclosure and the present invention or inventions and protected by the following claims.

1. A composition comprising a DNA repair modulator that specifically binds to the sequence KKYIEIRKEAREAANGDSDGPSYM (SEQ. ID NO.:16) and inhibits non-homologous end joining.

2. The composition of claim 1, wherein the DNA repair modulator comprises a polypeptide.

3. The composition of claim 2, wherein the polypeptide comprises the sequence QVKLQESGAELVKPGASVKLSCKAFDYTFTTYDINWIKQRPGQGLWIGWIYPGS GNNKYNEKFKGKATLTADKSSRAAYMHLSSLTSEDSAVYFCAGGPLNMTGFDY WGQGTTVTVSSDIELTQSPSSMYASLGERVTITCKASQDINSYLSWFQQKPGKSP KTLIYRANRLVDGVPSRFSGSGSGQDYSLTISSLEYEDMGIYYCLQYDELPLTFGA GTKLEIKR (SEQ. ID NO.:17).

4. The composition of claim 1, wherein less than about 50% of DNA-PKcs enzymatic activity is inhibit by the DNA repair modulator.

5. A single chain antibody that specifically binds to DNA-PKcs in a region outside of the catalytic domain, wherein the single chain antibody includes complementarity determining regions FTTYDIN (SEQ. ID NO.:18), WIYPGSGNNKYNEKFKG (SEQ. ID NO.:19), GPLNMTGFDY (SEQ. ID NO.:20), KASQDINSYLS (SEQ. ID NO.:21), RANRLVD (SEQ. ID NO.:22), and LQYDELPLT (SEQ. ID NO.:23), in an immunoglobin framework.

6. A pharmaceutical composition comprising an DNA repair modulator, a prodrug thereof, or combination thereof, wherein the modulator inhibits DNA repair by specifically interacting with DNA-PKcs outside of the DNA-PKcs catalytic domain.

7. The pharmaceutical composition of claim 6, wherein the DNA repair modulator comprises a single chain antibody.

8. The pharmaceutical composition of claim 6, wherein the DNA repair modulator interacts with a region of DNA-PKcs having the sequence KKKYIEIRKEAREAANGDSDGPSYM (SEQ. ID NO.:16).

9. The pharmaceutical composition of claim 6, wherein the DNA repair modulator inhibits DNA end joining.

10. The pharmaceutical composition of claim 8, wherein the DNA repair modulator comprises a single chain antibody.

11. The pharmaceutical composition of claim 6, wherein the DNA repair comprises a repair of a double-strand break.

12. The pharmaceutical composition of claim 6, further comprising a pharmaceutically acceptable carrier, excipient, or diluent.

13. A pharmaceutical composition comprising a DNA repair modulator, a prodrug thereof, or a combination thereof, wherein the modulator interacts with a DNA repair polypeptide and sterically inhibits the DNA repair polypeptide.

14. The pharmaceutical composition of claim 13, wherein the DNA repair modulator comprises a single chain antibody.

15. The pharmaceutical composition of claim 13, wherein the DNA repair modulator interacts with a region of DNA-PKcs.

16. The pharmaceutical composition of claim 15, wherein the region of DNA-PKcs include the sequence KKKYIEIRKEAREAANGDSDGPSYM (SEQ. ID. NO. 16) or a portion thereof.

17. The pharmaceutical composition of claim 13, wherein the DNA repair modulator inhibits DNA end joining.

18. The pharmaceutical composition of claim 15, wherein the DNA repair modulator comprises a single chain antibody.

19. The pharmaceutical composition of claim 13, further comprising a pharmaceutically acceptable carrier, excipient, or diluent.

20. A method of screening for DNA repair modulators comprising:
(a) introducing a test compound into a plurality of cells;
(b) inducing breaks in genetic material of the plurality of cells; and
(c) selecting the test compound that modulates the ability to repair breaks in the genetic material compared to the ability or repair breaks in genetic material of control cells and binds to DNA-PKcs in a region outside of DNA-PKcs's catalytic domain.

21. The method of claim 20, wherein the test compound that inhibits the ability to repair breaks in the genetic material is selected.

22. The method of claim 20, wherein the test compound that promotes the ability to repair breaks in the genetic material is selected.

23. The method of claim 20, wherein the test compound is introduced into the plurality of cells in vivo or in vitro.

24. A cell-free assay for identifying DNA repair modulators comprising the steps of:
(a) combining a test compound with a reaction mixture, wherein the reaction mixture comprises a DNA ligase IV/XRCC4 complex, optionally other DNA DSB repair proteins, introduced in purified form or as a mixture in a cell extract, and a plurality of oligonucleotides;
(b) comparing the presence of ligated oligonucleotides obtained from step (a) with ligated oligonucleotides from a control reaction mixture without the test compound;
(c) determining the effect of the test compound on DNA-dependent protein kinase activity; and
(d) selecting the test compound that results in fewer ligation products in step (a) than in a control reaction mixture without the test compound and does not completely inhibit DNA-dependent protein kinase activity.

25. The method of claim 24, wherein the oligonucleotides are labeled with a detectable marker.

26. The method of claim 24, wherein DNA-dependent protein kinase activity is determined by measuring the phosphorylation of p53 peptide.

27. A single chain antibody comprising an organelle localization signal sequence, wherein the single chain antibody inhibits DNA repair by binding to a DNA repair polypeptide.

28. The single chain antibody of claim 27, wherein the organelle localization signal is selected from the group consisting of a nuclear localization signal and a chloroplast localization signal.

29. The single chain antibody of claim 27, wherein the DNA repair polypeptide is DNA-PKcs.

30. The single chain antibody of claim 29, wherein the single chain antibody binds DNA-PKcs in a region outside the catalytic domain.

31. The single chain antibody of claim 30, wherein the region includes the sequence KKKYIEIRKEAREAANGDSDGPSYM (SEQ. ID NO.:16) or a portion thereof.

32. A single chain antibody comprising a protein transduction domain.

33. The single chain antibody of claim 32, wherein the single chain antibody binds to a DNA repair polypeptide.

34. The single chain antibody of claim 33, wherein the DNA repair polypeptide comprises DNA-PK.

35. The single chain antibody of claim 34, wherein the single chain antibody binds to a region of the DNA-PK polypeptide outside the catalytic domain.

36. The single chain antibody of claim 35, wherein the single chain antibody binds to a region including the sequence KKKYIEIRKEAREAANGDSDGPSYM (SEQ. ID NO.:16) or a portion thereof.

37. A pharmaceutical composition comprising a single chain antibody that binds to a polypeptide comprising the sequence KKKYIEIRKEAREAANGDSDGPSYM (SEQ. ID NO.:16).

38. A pharmaceutical composition of claim 37, wherein the single chain antibody comprises a protein transduction domain and an organelle localization signal.

39. The pharmaceutical composition of claim 38, wherein the organelle localization signal is selected from the group consisting of a nuclear localization signal and a chloroplast localization signal.

40. A vector comprising a promoter operably linked to a polynucleotide encoding a polypeptide comprising a single chain antibody that binds to DNA-PKcs in a region outside of the catalytic domain, a nuclear localization signal, and a protein transduction domain.

41. The vector of claim 40, wherein the promoter is inducible.

42. A method for treating cancer comprising:
introducing into a cancer cell a polypeptide comprising a single chain antibody that binds to DNA-PKcs in a region outside of the catalytic domain operably linked to a nuclear localization signal, wherein said polypeptide inhibits DNA end joining; and
exposing the cancer cell to an amount of ionizing radiation in an amount sufficient to induce breaks in the cancer cell's DNA.

43. A method for treating cancer comprising:
introducing into a cancer cell a polynucleotide encoding a single chain antibody that binds to DNA-PKcs in a region outside of the catalytic domain which is operably linked to a nuclear localization signal and a protein transduction domain, wherein said single chain antibody inhibits DNA end joining by binding DNA-PKcs; and
inducing DNA breaks in the cancer cell.

44. The method of claim 43, wherein the DNA breaks are induced by exposing the cancer cell to ionizing radiation.

45. A method for increasing radiation sensitivity of a cell, comprising:
introducing into the cell a DNA repair modulator, wherein the DNA repair modulator sterically inhibits a DNA repair polypeptide.

46. The method of claim 45, further comprising the step of exposing the cell to radiation.

47. The method of claim 46, wherein the radiation comprises ionizing radiation.

48. The method of claim 45, wherein the DNA repair modulator comprises a polypeptide.

49. The method of claim 48, wherein the polypeptide binds to DNA-PKcs.

50. The method of claim 49, wherein the polypeptide binds outside of the catalytic domain.

51. The method of claim 50, wherein the polypeptide binds to a region comprising the sequence KKKYIEIRKEAREAANGDSDGPSYM (SEQ. ID NO.:16) or a portion thereof.

52. A cell transfected with a vector comprising a promoter operably linked to a polynucleotide encoding a polypeptide comprising a single chain antibody that binds to DNA-PKcs.

53. The cell of claim 52, wherein the cell is stably transfected with the vector.

54. The cell of claim 52, wherein in the polynucleotide is stably integrated into the cell's genome.

55. The cell of claim 52, wherein the vector is episomal.

56. The cell of claim 52, wherein the promoter is inducible.

57. The cell of claim 52, wherein the polypeptide further comprises a nuclear localization signal.

58. The cell of claim 52, wherein the polypeptide binds to a second polypeptide comprising the sequence KKKYIEIRKEAREAANGDSDGPSYM (SEQ. ID NO.:16) or a portion thereof.

59. A method of sensitizing a cell to radiation comprising contacting the cell with a DNA repair modulator, wherein the DNA repair modulator combines with a DNA repair polypeptide to form an aggresome and thereby inhibits DNA repair.

60. A method for inducing cell death comprising contacting the cell with the composition of claim 1 and inducing cell death by exposing the cell to an amount of ionizing radiation sufficient to induce double-strand breaks in the cell's DNA.
